(19) United States
(12) Patent Application Publication
(10) Pub. No.: US 2008/0220084 A1
(43) Pub. Date: Sep. 11, 2008

(54) COMBINATION THERAPY FOR ALLEVIATING PAIN-RELATED CONDITIONS

(75) Inventors: Jian-Qiang FAN, Cresskill, NJ (US); Dawei Zhu, Shanghai (CN)

Correspondence Address: FOLEY AND LARDNER LLP;SUITE 500, 3000 K STREET NW, WASHINGTON, DC, 20007, US

(73) Assignee: Pfantastic Medical Research Institute, LLC

(21) Appl. No.: 12/044,763

(22) Filed: Mar. 7, 2008

Related U.S. Application Data
(60) Provisional application No. 60/894,139, filed on Mar. 9, 2007.

(51) Int. Cl. A61K 35/64; A61K 35/12; A61K 35/56; A61K 35/58; A61K 31/192; A61K 36/00; A61P 25/06

(52) U.S. Cl. 424/538; 424/520; 424/542; 424/547; 424/725; 514/569; 424/773

(57) ABSTRACT

The present invention provides compositions and methods for alleviating physical discomfort and/or pain-related conditions in a subject, comprising one or more ingredients that improve blood circulation, one or more ingredients that reduce pain and one or more ingredients that enhance bone health. The ingredients are selected from synthetic compounds, natural products, natural ingredients, extracts from natural ingredients, or combinations thereof. The compositions of the invention are particularly useful in alleviating musculoskeletal pain and nerve related pain.

### CROSS-REFERENCE TO RELATED PATENT APPLICATIONS

This application claims priority from U.S. Provisional Application No. 60/894,139, filed Mar. 9, 2007, incorporated herein by reference in its entirety.

### FIELD OF THE INVENTION

The present invention provides compositions, pharmaceutical formulations and herbal formulas comprising ingredients selected from synthetic compounds, natural products, natural ingredients, extracts from natural ingredients, or combinations thereof. The present invention also provides methods of use and process of making the same.

### BACKGROUND

Musculoskeletal pain is of epidemic proportions in America, with 83 million adults living with pain that affects their participation in daily activities, and 75 million people with chronic debilitating pain. For example, as many as 1 in 3 adults in the United States currently suffer from chronic joint symptoms or arthritis. Osteoarthritis (OA) and rheumatoid arthritis (RA) are two of the common forms, in which approximately 21 million and 2.1 million adults are affected by OA and RA, respectively, in the United States. Low back pain can cost the nation an estimated $27 billion annually in medical claims, and the same in disability payments and lost productivity.

Despite the wide spread diseases or conditions, therapeutic options are limited and effectiveness of therapies remains insufficient. Non-steroidal anti-inflammatory drugs (NSAIDs) can be used to reduce acute inflammation thereby decreasing pain and improving function. NSAIDs can include aspirin, sulindac, COX-2 selective inhibitor drugs, etc. NSAIDs may not be efficient enough in controlling pain involving joints and nerve tissues because of difficulty to deliver them through soft tissues between joints. Additionally, a large dose of NSAIDs may increase serious cardiovascular and gastrointestinal risks. Corticosteroids that can be given systemically or can be injected intra-articularly, can be useful in early stages of the disease as temporary adjunctive therapy. Physical therapies can be effective in temporary relief of pain. In some cases, traction involving the use of weights to apply constant or intermittent force to gradually pull the skeletal structure into better alignment can be helpful. Another option can be an invasive surgical intervention. Therefore, alternative treatment for musculoskeletal or nerve pain-related disorders/conditions which can be efficient, non-invasive and has lesser side effects, is desirable.

Traditional Chinese medicine (TCM) has been used for achieving various therapeutic benefits. TCM comprises natural ingredients obtained from herbs, animal parts, insects that produce and contain a variety of chemical substances acting upon the body to accomplish therapeutic effects. TCM have been documented back to thousand's year ago. Among them, Shen Nong Ben Cao Jing is believed to be documented in Han Dynasty of China (206 BC-AD 220). The Compendium of Materia Medica (Ben Cao Gang Mu) is a pharmaceutical text written by Li Shizhen (1518-1593 AD) during the Ming Dynasty of China. The text lists the animals, plants and other objects which can contain medicinal properties.

Although the ingredients in TCM have beneficial effects, a well balanced and synergized combination of the ingredients can be used to maximize the therapeutic effectiveness and improving the overall health of an individual. The present invention provides compositions, pharmaceutical formulations, herbal formulas, and methods of use comprising ingredients selected from synthetic compounds, natural products, natural ingredients, extracts from natural ingredients, or combinations thereof for alleviating musculoskeletal and nerve-related pains.

### SUMMARY OF THE INVENTION

First aspect of the present invention provides a composition useful for alleviating physical discomfort in a subject, comprising one or more ingredients that improve blood circulation, one or more ingredients that reduce pain and one or more ingredients that enhance bone health.

Second aspect of the invention provides a composition useful for alleviating a pain-related condition in a subject, comprising one or more ingredients that improve blood circulation, one or more ingredients that reduce pain and one or more ingredients that enhance bone health.

Third aspect of the invention provides a composition useful for alleviating a pain-related condition in a subject, comprising one or more ingredients or extract therefrom selected from Rhizoma Gastrodiae Elatae, Squama Manitis, Agkistrodon seu Bungarus, Radix Aconiti Lateralis Preparata, Radix Paeoniae Lactiflorae, Herba Asari cum Radice, Cornu cervi Parvum, or Eupolyphaga seu Opisthoplatia.

Fourth aspect of the invention provides a composition useful for alleviating a pain-related condition in a subject, comprising one or more ingredients or extract therefrom selected from Rhizoma Gastrodiae Elatae, Lumbricus, Agkistrodon seu Bungarus, Radix Aconiti Lateralis Preparata, Radix Paeoniae Lactiflorae, Herba Asari cum Radice, Cornu cervi Parvum, or Eupolyphaga seu Opisthoplatia.

Fifth aspect of the invention provides a process of preparing a composition useful for alleviating physical discomfort in a subject, comprising: combining:

one or more ingredients that improve blood circulation selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba or extracts therefrom;

one or more ingredients that reduce pain selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, or extracts therefrom; and

one or more ingredients that enhance bone health selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, glucosamine, acetylglucosamine, chondroitin, calcium, phosphorus, or extracts from natural ingredients.

Sixth aspect of the invention provides a process of preparing a composition useful for alleviating a pain-related condition in a subject, comprising combining:

one or more ingredients that improve blood circulation selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba, xanthinol nicotinate, 3,5-pyridinedicarboxylic acid, amlodipine, prazosin, doxazosin, nitroglycerin, sildenafil, L-arginine, or extracts from natural ingredients;

one or more ingredients that reduce pain selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, morphine, diamorphine, fentanyl, buprenorphine, oxycodone, codeine, aspirin, ibuprofen, diclofenac, celecoxib, or extracts from natural ingredients; and

one or more ingredients that enhance bone health elected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, bisphosphonates, glucosamine, acetylglucosamine, chondroitin, calcium, phosphorus, or extracts from natural ingredients.

Seventh aspect of the invention provides a process of preparing a composition useful for alleviating a pain-related condition in a subject, comprising combining Rhizoma Gastrodiae Elatae, Squama Manitis, Agkistrodon seu Bungarus, Radix Aconiti Lateralis Preparata, Radix Paeoniae Lactiflorae, Herba Asari cum Radice, Cornu cervi Parvum, Eupolyphaga seu Opisthoplatia, extracts therefrom, or combinations thereof.

Eighth aspect of the invention provides a process of preparing a composition useful for alleviating a pain-related condition in a subject, comprising combining Rhizoma Gastrodiae Elatae, Lumbricus, Agkistrodon seu Bungarus, Radix Aconiti Lateralis Preparata, Radix Paeoniae Lactiflorae, Herba Asari cum Radice, Cornu cervi Parvum, Eupolyphaga seu Opisthoplatia, extracts therefrom, or combinations thereof.

Ninth aspect of the invention provides a method of improving an efficacy of one or more non-steroidal anti-inflammatory drugs (NSAIDs) in a subject, comprising co-administering with the one or more NSAIDs an effective amount of a formulation to a subject, the formulation comprising:

(a) a combination of one or more ingredients that improve blood circulation selected from selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba or extracts therefrom;

one or more ingredients that reduce pain selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, or extracts therefrom; and

one or more ingredients that enhance bone health selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, glucosamine, acetylglucosamine, chondroitin, calcium, phosphorus, or extracts from natural ingredients; and

b) one or more of NSAIDs.

Tenth aspect of the invention provides a method of improving an efficacy of one or more non-steroidal anti-inflammatory drugs (NSAIDs) in a subject, comprising co-administering with the one or more NSAIDs an effective amount of a formulation to a subject, the formulation comprising (a) a combination of ingredients or extracts therefrom comprising Rhizoma Gastrodiae Elatae, Squama Manitis, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia; and (b) one or more of NSAIDs.

Eleventh aspect of the invention provides a method of improving an efficacy of one or more non-steroidal anti-inflammatory drugs (NSAIDs) in a subject, comprising co-administering with the one or more NSAIDs an effective amount of a formulation to a subject, the formulation comprising (a) a combination of ingredients or extracts therefrom comprising Rhizoma Gastrodiae Elatae, Lumbricus, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia; and (b) one or more of NSAIDs.

Twelfth aspect of the invention provides a method of alleviating physical discomfort in a subject comprising: administering to a subject an effective amount of a formulation comprising a combination of:

one or more ingredients that improve blood circulation selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba or extracts therefrom;

one or more ingredients that reduce pain selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, or extracts therefrom; and

one or more ingredients that enhance bone health selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, glucosamine, acetylglucosamine, chondroitin, calcium, phosphorus, or extracts from natural ingredients.

Thirteenth aspect of the invention provides a method of alleviating a pain-related condition in a subject comprising administering to a subject an effective amount of a formulation comprising a combination of

one or more ingredients that improve blood circulation selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba, xanthinol nicotinate, 3,5-pyridinedicarboxylic acid, amlodipine, prazosin, doxazosin, nitroglycerin, sildenafil, L-arginine, or extracts from natural ingredients;

one or more ingredients that reduce pain selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, morphine, diamorphine, fentanyl, buprenorphine, oxycodone, codeine, aspirin, ibuprofen, diclofenac, celecoxib, or extracts from natural ingredients; and

one or more ingredients that enhance bone health selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, bisphosphonates, glucosamine, acetylglucosamine, chondroitin, calcium, phosphorus, or extracts from natural ingredients.

Fourteenth aspect of the invention provides a method of alleviating a pain-related condition in a subject comprising administering to a subject an effective amount of a formulation comprising a combination of ingredients or extracts therefrom comprising Rhizoma Gastrodiae Elatae, Squama Manitis, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia.

Fifteenth aspect of the invention provides a method of alleviating a pain-related condition in a subject comprising administering to a subject an effective amount of a formulation comprising a combination of ingredients or extracts therefrom comprising Rhizoma Gastrodiae Elatae, Lumbricus, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia.

Sixteenth aspect of the invention provides a herbal formula that promotes cardiovascular function and a healthy circulatory system, supports cartilage and joint function, strengthens bone health, and alleviates muscle fatigue comprising:

one or more ingredients or extracts therefrom selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, or Ginko biloba;

one or more ingredients or extracts therefrom selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, or Rhizoma Gastrodiae Elatae; and

one or more ingredients or extracts therefrom selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, glucosamine, acetylglucosamine, or chondroitin.

Seventeenth aspect of the invention provides a herbal formula that promotes cardiovascular function and a healthy circulatory system, supports cartilage and joint function, strengthens bone health, and alleviates muscle fatigue, comprising a combination of ingredients or extracts therefrom comprising Rhizoma Gastrodiae Elatae, Squama Manitis, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia.

Eighteenth aspect of the invention provides a herbal formula that promotes cardiovascular function and a healthy circulatory system, supports cartilage and joint function, strengthens bone health, and alleviates muscle fatigue, comprising a combination of ingredients or extracts therefrom comprising Rhizoma Gastrodiae Elatae, Lumbricus, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia.

Nineteenth aspect of the invention provides a pharmaceutical composition for alleviating a pain-related condition in a subject, comprising a combination of:

(a) one or more ingredients that improve blood circulation selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba, xanthinol nicotinate, 3,5-pyridinedicarboxylic acid, amlodipine, prazosin, doxazosin, nitroglycerin, sildenafil, L-arginine, or extracts from natural ingredients;

one or more ingredients that reduce pain selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, morphine, diamorphine, fentanyl, buprenorphine, oxycodone, codeine, aspirin, ibuprofen, diclofenac, celecoxib, or extracts from natural ingredients; and

one or more ingredients that enhance bone health selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, bisphosphonates, glucosamine, acetylglucosamine, chondroitin, calcium, phosphorus, or extracts from natural ingredients; and (b) one or more of NSAIDs.

Twentieth aspect of the invention provides a pharmaceutical composition for alleviating a pain-related condition in a subject, comprising (a) a combination of ingredients or extracts therefrom comprising Rhizoma Gastrodiae Elatae, Squama Manitis, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia, and (b) one or more of NSAIDs.

Twentyfirst aspect of the invention provides a pharmaceutical composition for alleviating a pain-related condition in a subject, comprising (a) a combination of ingredients or extracts therefrom comprising Rhizoma Gastrodiae Elatae, Lumbricus, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia; and (b) one or more of NSAIDs.

Twentysecond aspect of the invention provides a method of alleviating osteoarthritis in a subject comprising: administering to a subject an effective amount of a formulation comprising a combination of:

one or more ingredients that improve blood circulation selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba, xanthinol nicotinate, 3,5-pyridinedicarboxylic acid, amlodipine, prazosin, doxazosin, nitroglycerin, sildenafil, L-arginine, or extracts from natural ingredients;

one or more ingredients that reduce pain selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, morphine, diamorphine, fentanyl, buprenorphine, oxycodone, codeine, aspirin, ibuprofen, diclofenac, celecoxib, or extracts from natural ingredients; and

one or more ingredients that enhance bone health selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, bisphosphonates, glucosamine, acetylglucosamine, chondroitin, calcium, phosphorus, or extracts from natural ingredients.

Twentythird aspect of the invention provides a method of alleviating rheumatoid arthritis in a subject comprising: administering to a subject an effective amount of a formulation comprising a combination of:

one or more ingredients that improve blood circulation selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba, xanthinol nicotinate, 3,5-pyridinedicarboxylic acid, amlodipine, prazosin, doxazosin, nitroglycerin, sildenafil, L-arginine, or extracts from natural ingredients;

one or more ingredients that reduce pain selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, morphine, diamorphine, fentanyl, buprenorphine, oxycodone, codeine, aspirin, ibuprofen, diclofenac, celecoxib, or extracts from natural ingredients; and

one or more ingredients that enhance bone health selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, bisphosphonates, glucosamine, acetylglucosamine, chondroitin, calcium, phosphorus, or extracts from natural ingredients.

Twentyfourth aspect of the invention provides a method of alleviating cervical spondylosis in a subject comprising: administering to a subject an effective amount of a formulation comprising a combination of:

one or more ingredients that improve blood circulation selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba, xanthinol nicotinate, 3,5-pyridinedicarboxylic acid, amlodipine, prazosin, doxazosin, nitroglycerin, sildenafil, L-arginine, or extracts from natural ingredients;

one or more ingredients that reduce pain selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, morphine, diamorphine, fentanyl, buprenorphine, oxycodone, codeine, aspirin, ibuprofen, diclofenac, celecoxib, or extracts from natural ingredients; and

one or more ingredients that enhance bone health selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, bisphosphonates, glucosamine, acetylglucosamine, chondroitin, calcium, phosphorus, or extracts from natural ingredients.

### BRIEF DESCRIPTION OF THE DRAWINGS

FIG. 1 illustrates a self-diagnosis of physical discomfort in a subject after administration of the formulation of the invention as in Example 1.

FIG. 2 illustrates a self-diagnosis of physical discomfort in a subject after administration of the formulation of the invention as in Example 3.

### DETAILED DESCRIPTION OF THE INVENTION

### Definitions

The term “alleviating” and its grammatical equivalents as used herein include achieving a beneficial effect, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. The term, “alleviating” is not intended to require complete reduction in a condition being considered such as, pain related condition. Such reduction is by at least about 50%, at least about 75%, at least about 90%, at least about 95%, or at least about 99.9% of the pain in a pain related condition. The term refers to an observable or measurable reduction in the pain related condition. In treatment scenarios, the alleviation is sufficient to produce a therapeutic and/or prophylactic benefit in the condition being treated.

The term, “composition” or “formulation” is used interchangeably herein.

The term “pharmaceutically acceptable excipient” as used herein, means those carriers which retain the biological effectiveness and properties of the ingredients of the present invention, and which are not biologically or otherwise undesirable.

The term “subject” or its grammatical equivalents as used herein refers to a warm-blooded animal such as a mammal who is healthy or is afflicted with, or suspected to be afflicted with a disease. Preferably, “subject” refers to a human. The subject can also be an animal, such as, domestic animals (e.g., dogs, cats and the like), farm animals (e.g., cows, sheep, pigs, horses and the like) and laboratory animals (e.g., rats, mice, guinea pigs and the like).

### A. Compositions

One aspect of the invention provides compositions useful for alleviating physical discomfort in a subject using ingredients that can enhance beneficial effects in the subject including, but are not limited to, improving blood circulation, reducing pain and enhancing bone health.

Increase of blood circulation can be achieved by improving cardiovascular function, expanding coronary artery, strengthening coronary blood flow, expanding peripheral blood vessels, removing blood stasis, or combination thereof. Improved blood circulation itself can reduce inflammation. Improved blood circulation can also increase blood flow to tissues, such as bone tissues and surrounding soft tissues, and nerve tissues, that are normally difficult to be accessed. A combination of ingredients that increase blood circulation and reduce pain permits quick delivery of active ingredients that reduce pain to various soft tissues and nerve tissues, thus providing a better tissue penetration. Increased blood circulation also accelerates the delivery of effective ingredients that stimulate bone health for a long term curative effective associated with bone-related disorders or conditions. The synergy, generated from the combination of the ingredients that improve blood circulation, reduce pain and enhance bone health, can yield unexpected therapeutic effects for alleviating physical discomfort and pain-related conditions, as described in this invention.

The present invention provides compositions and methods for pharmaceutical use as well as a nutraceutical use in a subject. The subject is preferably a human. A pharmaceutical can be a composition that is used as a medicament to cure a disease condition with or without a physician's prescription. A nutraceutical can be a composition that can be used as a dietary supplement for promoting general health or to remedy a particular condition of the body. Nutraceuticals can be sold in health food stores and supplied without a physician's prescription.

In a particular embodiment of the aforementioned aspect of the invention, the invention provides a composition useful for alleviating physical discomfort in a subject, comprising one or more ingredients that improve blood circulation, one or more ingredients that reduce pain and one or more ingredients that enhance bone health. In one embodiment, the ingredients that can be used in the compositions useful for alleviating physical discomfort in a subject, include without limitation, natural products, natural ingredients, extracts from natural ingredients, or combinations thereof. In one embodiment, the compositions useful for alleviating physical discomfort in a subject can be used as a dietary supplement.

The examples of physical discomfort that can be alleviated by the compositions of the present invention include, but are not limited to, headache, migraine, postoperative pain, ear pain, throat pain, eye pain, cutaneous pain, mild-to-moderate pain due to inflammation of tendons or ligaments, tissue injury, pain due to sports injury or strain, pyrexia, sciatic nerve pain, renal colic, hardness and stiffness of shoulder, cold constitutions, edema, frequent tiredness, insomnia, sleep difficulty due to pains, depression, dysmenorrhea, menstruation pain, amenorrhea, menopausal syndrome, muscle weakness, reduced blood circulation, weak bones, or combinations thereof.

Another aspect of the invention provides a composition useful for alleviating a pain-related condition in a subject, comprising one or more ingredients that improve blood circulation, one or more ingredients that reduce pain and one or more ingredients that enhance bone health. Such composition contain one or more ingredients selected from synthetic compounds, natural products, natural ingredients, extracts from natural ingredients, or combinations thereof. In on embodiment, the pain-related conditions that can be alleviated by the compositions of the present invention include, but are not limited to, musculoskeletal pain, nerve related pain, arthritis, rheumatoid arthritis, osteoarthritis, polyarthritis, ulnar deviation, boutonniere deformity, swan neck deformity, z-thumb deformity, subluxation at the metacarpophalangeal joint, inflammatory arthropathies, cervical spondylosis, ankylosing spondylitis, psoriatic arthritis, Reiter's syndrome, gout, metastatic bone pain, cancer pain, herniated disc, tempromandibular joint dysfunction, tendonitis, torticollis, trigeminal neuralgia, cervical syndrome, thoracic outlet syndrome, complex regional pain syndrome, or combinations thereof.

Yet another aspect of the present invention provides a herbal formula that promotes cardiovascular function and a healthy circulatory system, supports cartilage and joint function, strengthens bone health, and alleviates muscle fatigue. In a particular embodiment, the herbal formula comprises:

one or more ingredients or extracts therefrom selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, or Ginko biloba;

one or more ingredients or extracts therefrom selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, or Rhizoma Gastrodiae Elatae; and

one or more ingredients or extracts therefrom selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, glucosamine, acetylglucosamine, or chondroitin.

In one preferred embodiment, the herbal formula that promotes cardiovascular function and a healthy circulatory system, supports cartilage and joint function, strengthens bone health, and alleviates muscle fatigue, comprises a combination of ingredients or extracts therefrom comprising Rhizoma Gastrodiae Elatae, Squama Manitis, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia.

In another preferred embodiment, the herbal formula that promotes cardiovascular function and a healthy circulatory system, supports cartilage and joint function, strengthens bone health, and alleviates muscle fatigue, comprises a combination of ingredients or extracts therefrom comprising Rhizoma Gastrodiae Elatae, Lumbricus, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia.

a. Ingredients

Table 1 provides a list of natural ingredients or extract therefrom that can be used in the present invention for enhancing blood circulation, reducing pain, and increasing bone health. The administration of the combination of these ingredients can improve the efficacy of individual ingredients and can help in improving the overall health of the individual.

TABLE 1
List of Natural Ingredients

| English name | Pharmaceutical name | Botanical name |
|---|---|---|
| Chi Jian | Rhizoma Gastrodiae Elatae | Gastrodia elata Bl. |
| Pangolin Scales | Squama Manitis | Manitis pentadactyla Linnaeus |
| Bungarus Parvus, or | Agkistrodon seu Bungarus | Bungarus multicinctus Blyth |
| Agkistrodon Acutus | | |
| Prepared roots of | Radix Aconiti Lateralis | Acontium carmichaeli Debx., A. Kusnezoffii |
| common Monkshood | Preparata | Reichb. |
| Chinese rat snake | Zaocys dhumnades | Zaocys dhumnades Cantor |
| Earthworm | Lumbricus | Allolobophora calinosa (Savigny) |
| | | trapezoides (Ant. Duges), Pheretima |
| | | asiatica, P. aspergillum (Perricr) |
| Chinese Angelica | Radix Angelicae sinensis | Angelica sinensis (Oilv.) Diels |
| root | | |
| Suberect Spatholobus | Radix et Caulis Jixuetent | Millettia dielsiana Harms, M. reticulata |
| Stem | | Benth. |
| Prepared Rhizome of | Radix Rehmanniae | Rehmannia glutinosa (Gaertn.) Libosch |
| adhesive Rehmannia | Glutinosae Conquitae | |
| Lobed Kudzuvine | Radix Puerariae | Pueraria lobota (Willd) Ohwi. |
| root | | |
| Tuber fleeceflower | Caulis Polygoni Multiflori | Polygonum multiflorum Thunb. |
| stem and leaf | | |
| Chinese Clematis | Radix Clemetidis Chinesis | Clematis chinesis Osbeck |
| Root | | |
| Common | Radix Tripterygium wilfordii | Tripterygium wilfordii Hook. f. |
| threewingnut root | | |
| and leaf | | |
| Prepared mother and | Radix Tripterygium | Tripterygium wilfordii Hook. f. |
| daughter root of | willfordii | |
| common Monkshood | | |
| Manchurian | Herba Asari cum Radice | Asarum heterotropoides Fr. Schmidt |
| wildginger herb | | var. mandshuricum (Maxim.) Kitag; |
| | | A. sieboldi Mig |
| Peony root | Radix Paeoniae Lactiflorae | Paeonia lactiflora Pall. |
| Centipede | Scolopendra Subspinipes | Scolopendra subspinipes Leach |
| Scorpion | Buthus Martensi | Buthus martensii Karsch. |
| Myrrh | Myrrha | Commiphora myrrha Engl., |
| | | Balsamodendron ehrenbergianum Berg. |
| Common Clubmoss | Herba Lycopodii | Lycopodium japonicum Thumb. |
| herb | | |
| Olibanum | Gummi Olibanum | Boswellia carterii Birdw. |
| Frankincense | | |
| Eucommia bark | Cortex Eucommiae Ulmoidis | Eucommia ulmoide Oliv. |
| Szechwan Lovage | Radix Ligustici Wallichii | Ligusticum wallichii Franch. |
| Rhizome | | |
| Sanchi Root | Radix Pseudoginseng | Panax pseudoginseng Wall. Var. |
| | | notoginseng (Burkill) |
| Gastrodia Tuber | Rhizoma Gastrodiae Elatae | Gastrodia elata Bl., |
| Wingless cockroach, | Eupolyphaga seu | Eupolyphaga sinensis Walker; |
| eupolyphaga | Opisthoplatia | Opsithoplatia orientalis Burmeiter |
| Velvet of young deer | Cornu cervi Parvum | Cervus elaphus Linnaeus; C. nipoon |
| antler or deer antler | | Temminck |
| (lu jiao) | | |
| Drynaria rhizome | Rhizoma Drynariae | Drynaria fortunei (Kunze) J. Sm, |
| | | Drynaria baronii (Christ) Diels |
| Shorthorned | Herba Epimedii | Epimedium grandiflorum Morr., E. Sagittatum |
| Epimedium Herb | | Maxim. |
| Chinese Pyrola Herb | | Pyrola calliantha H. Andres |
| Fleece flower root | Radix Polygoni Multiflori | Polygonum multiflorum Thunb. |
| Rehmannia | Epimedium macranthum, | Rehmannia glutinosa Libosch |
| | Morr et Dcene., Epimedium | |
| | sagittatum, Bak. | |
| Tubiechong | Eupolyphaga seu | Blattaria corydiidae Polyphaginae. |
| | Opisthoplatia | |
| Malaytea Scurfpea | Fructus Psoraleae Corylifoliae | Psoralea corylifolia L. |
| Fruit | | |

One or more of the natural ingredients listed in Table 1 can be used in the compositions of the present invention for various beneficial effects. Examples of such beneficial effects for some of these natural ingredients are as follows. Rhizoma Gastrodiae Elatae (Gastrodia elata Bl.) is a traditional Chinese medicine that can be used for alleviating various conditions including but are not limited to, epilepsy, external injury of the brain, pain, neurasthenia, depression, and/or the blood-vessel type of migraine. Rhizoma Gastrodiae Elatae can be used for relieving convulsion and spasm; suppressing symptoms of liver-wind; calming the liver; reducing symptoms such as vertigo and headache; removing obstruction in the channels to relieve pain; arthralgia due to the wind, cold and/or dampness, or numbness and paralysis caused by liver; and/or neurasthenia and insomnia. Rhizoma Gastrodiae Elatae can have effect on the CNS such as, sedation, anti-seizure, and/or painkiller; effect on heart and blood vessels such as, protecting the heart muscle from lacking circulation, improving blood flow in the coronary artery; improving blood flow to the brain; mitigating the effect of adrenalin on blood vessel constriction, and/or lower blood pressure; effect on tolerance of lack of oxygen; and/or effect on improving the immune system. Other effects of Rhizoma Gastrodiae Elatae include, but are not limited to, increasing the skin temperature; increasing the expansion and contraction ability of the smooth muscle of the intestine; improving the secretion of the bile in the gall bladder; and/or slowing down breathing. Rhizoma Gastrodiae Elatae can be administered as a decoction or as a powder.

Squama Manitis (Manitis pentadactyla Linnaeus), can improve blood flow in the coronary artery, help in promoting lactation, hastening boils to ripen, expelling pus, stopping pain, reducing pain in the joints of lower limbs, chronic malaria, getting rid of parasites, unblocking menstruation, and/or undo blood stasis.

Agkistrodon seu Bungarus extract is the dried body of Bungarus multicinctus multicinctus Blyth (Fam. Elapidae). Agkistrodon seu Bungarus can be used for promoting collateral circulation, expanding blood vessels, anticoagulation, and treating chronic rheumatoid arthritis with numbness and muscula contracture; apoplectic hemiplegia and facial paralysis; convulsion, tetanus; and festered scrofula. It can be grounded into powder and taken with water.

Radix Aconiti Lateralis Preparata is obtained form a common monkshood mother root. Radix Aconiti Lateralis Preparata helps in expelling pain due to wind and cold. Radix Aconiti Lateralis Preparata can be collected from daughter root, rootlet and soil, and dried in the sun. The roots can also be processed to obtain the ingredient.

Roots of Paeonia lactiflora Pall. contain paeoniflorin, which can have a antispasmodic effect on mammalian intestines, reduce blood pressure, reduce body temperature caused by fever and protect against stress ulcers. It can be taken internally in the treatment of menstrual disorders, injuries, high blood pressure, pre-menstrual tension and liver disorders. The roots can be harvested in the autumn from cultivated plants and can be boiled before being sun-dried for later use. The roots of wild plants can also be harvested in the spring or in the autumn and can be sun-dried for later use. A tea made from the dried crushed petals of various peony species can be used as a cough remedy, and as a treatment for haemorrhoids and varicose veins. The roots of Paeonia lactiflora Pall. can be used as an alternative (a gradual beneficial change in the body), analgesic, anodyne, antibacterial, anti-inflammatory, antiseptic, antispasmodic, astringent, carminative, diuretic, emmenagogue, expectorant, febrifuge, hypotensive, nervine and tonic.

Herba Asari cum Radice can be used for various conditions, including but are not limited to, vertigo and headache; toothache due to wind and cold; arthralgia due to wind, cold and dampness; cough and dyspnea with retention of fluid; sinusitis marked by obstruction of the nose with headache, and episodes of running nose; and/or locally for the treatment of aphtha and tongue sore. Herba Asari cum Radice can help to dispel wind-cold, relieve nasal obstruction, alleviate pain and remove retained fluid. Herba Asari cum Radice can be used as a powder or a decoction.

Eupolyphaga seu Opisthoplatia is one of the TCM substances for efficiently penetrating through soft tissues to joints, nurturing bone tissues and enhancing bone health. It can be used for injuries of bones and muscles from impact, sprain in low back area. It can also be used for invigorating blood thereby breaking blood stasis.

The compositions for alleviating physical discomfort and pain-related conditions can also comprise natural products. The examples of natural products used as ingredients in the present invention include, but are not limited to, Hydergine, Vincamine, Nimodipine, Vinpocetine, Ginko biloba, glucosamine, acetylglucosamine, chondroitin, calcium, and/or phosphorus.

Hydergine is a mixture of alkaloids that come from a fungus (ergot) that grows on rye. Hydergine increases blood circulation. Additionally, Hydergine can have antioxidant and nerve growth factor (NGF)-like properties. Vincamine, Nimodipine, and Vinpocetine are all obtained from periwinkle and can promote brain blood circulation. Ginko biloba can increase brain blood circulation.

The compositions for alleviating pain-related conditions can also comprise synthetic compounds. The examples of synthetic compounds used as ingredients in the present invention include, but are not limited to, xanthinol nicotinate, 3,5-pyridinedicarboxylic acid (nifedipine, calcium channel blocker), amlodipine (Norvasc), prazosin (Minipres), doxazosin (Cardura, both α-receptor blockers), nitroglycerin, sildenafil (Viagra), L-arginine, morphine, diamorphine, fentanyl, buprenorphine, oxycodone, codeine, aspirin, ibuprofen, diclofenac, celecoxib, and/or bisphosphonates.

Bisphosphonates belong to a class of compounds similar to pyrophosphate. In bisphosphonates, the oxygen atom of the pyrophosphate is replaced by a carbon atom resulting in a P—C—P bond. Bisphosphonates exert a potent inhibitory effect on osteoclasts and can therefore be potent antiresorptive agents. Bisphosphonates can reduce bone turnover, increase bone mineral density, and decrease fracture risk such as, at the lumbar spine and the hip.

In one particular embodiment of the invention, the one or more ingredients that improve blood circulation used in the compositions for alleviating physical discomfort, are selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba, or extracts therefrom.

In another embodiment, the one or more ingredients that reduce pain used in the compositions for alleviating physical discomfort, are selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, or extracts therefrom.

In yet another embodiment, the one or more ingredients that enhance bone health used in the compositions for alleviating physical discomfort, are selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, glucosamine, acetylglucosamine, chondroitin, calcium, phosphorus, or extracts from natural ingredients.

In one particular embodiment of the invention, the composition for alleviating a pain-related condition in a subject, comprises:

one or more ingredients that improve blood circulation selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba, xanthinol nicotinate, 3,5-pyridinedicarboxylic acid, amlodipine, prazosin, doxazosin, nitroglycerin, sildenafil, L-arginine, or extracts from natural ingredients;

one or more ingredients that reduce pain selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, morphine, diamorphine, fentanyl, buprenorphine, oxycodone, codeine, aspirin, ibuprofen, diclofenac, celecoxib, or extracts from natural ingredients; and

one or more ingredients that enhance bone health selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, bisphosphonates, glucosamine, acetylglucosamine, chondroitin, calcium, phosphorus, or extracts from natural ingredients.

In one preferred embodiment, the invention provides a composition useful for alleviating a pain-related condition in a subject comprising one or more ingredients or extract therefrom selected from Rhizoma Gastrodiae Elatae, Squama Manitis, Agkistrodon seu Bungarus, Radix Aconiti Lateralis Preparata, Radix Paeoniae Lactiflorae, Herba Asari cum Radice, Cornu cervi Parvum, or Eupolyphaga seu Opisthoplatia.

In another preferred embodiment, the invention provides a composition useful for alleviating a pain-related condition in a subject, comprising one or more ingredients or extract therefrom selected from Rhizoma Gastrodiae Elatae, Lumbricus, Agkistrodon seu Bungarus, Radix Aconiti Lateralis Preparata, Radix Paeoniae Lactiflorae, Herba Asari cum Radice, Cornu cervi Parvum, or Eupolyphaga seu Opisthoplatia.

b. Formulations

In preparing a suitable combination of the ingredients, the ailment and the constitutional body type of the individual can be taken into account for treatment. The combination of the ingredients can be tailored to the individual human or an animal, to alleviate their pain-related condition or physical discomfort, enhance their recuperative power and state of well-being. The herbal formulation of the invention can supplement the human's or animal's diet and fortify the constitution as well as prevent or remedy ailments.

Without limiting the scope of the present invention, various examples of the formulation (#1-21) comprising combination of the ingredients that can be used in the present invention, are as shown below in Table 2.

TABLE 2
Examples of the Formulations Containing Combination of the
Ingredients

| | FORMULATION # | | | | | | | | | | | | | | | | | | | | |
|---|---|---|---|---|---|---|---|---|---|---|---|---|---|---|---|---|---|---|---|---|---|
| INGREDIENTS | 1 | 2 | 3 | 4 | 5 | 6 | 7 | 8 | 9 | 10 | 11 | 12 | 13 | 14 | 15 | 16 | 17 | 18 | 19 | 20 | 21 |
| Squama Manitis | x | | | | x | | | x | x | x | | | x | x | | | x | x | | | |
| Agkistrodon | x | x | x | | | | | | | | | | | | | x | | | | x | |
| seu Bungarus | | | | | | | | | | | | | | | | | | | | | |
| Zaocys | | | | | | | | | | | | | | x | x | | | | x | | |
| dhumnades | | | | | | | | | | | | | | | | | | | | | |
| Lumbricus | | x | x | | | | | | x | | x | x | | | | | x | | x | x | x |
| Radix | | | | x | | x | | | x | x | | x | x | | x | x | | x | x | | x |
| Angelicae | | | | | | | | | | | | | | | | | | | | | |
| sinensis | | | | | | | | | | | | | | | | | | | | | |
| Radix et Caulis | | | | x | | | | x | | x | | x | x | | | x | x | | x | | |
| Jixuetent | | | | | | | | | | | | | | | | | | | | | |
| Radix | | | | | | | x | | x | | x | | | | x | | | x | | | |
| Rehmanniae | | | | | | | | | | | | | | | | | | | | | |
| Glutinosae | | | | | | | | | | | | | | | | | | | | | |
| Conquitae | | | | | | | | | | | | | | | | | | | | | |
| Radix Puerariae | | | | x | x | x | x | x | x | x | x | | | | | | | | | | |
| Caulis Polygoni | | | | | | | | | | | | | | x | | | | | | x | |
| Multiflori | | | | | | | | | | | | | | | | | | | | | |
| Radix | | | | | | x | x | x | x | x | x | x | | | | x | | | x | | x |
| Clemetidis | | | | | | | | | | | | | | | | | | | | | |
| Chinesis | | | | | | | | | | | | | | | | | | | | | |
| Radix | | | | | | | | | | | | | | | x | | | | | x | |
| Tripterygium | | | | | | | | | | | | | | | | | | | | | |
| wilfordii | | | | | | | | | | | | | | | | | | | | | |
| Radix Aconiti | x | x | x | | | x | | | | | | x | x | | | | x | x | x | x | |
| Lateralis | | | | | | | | | | | | | | | | | | | | | |
| Preparata | | | | | | | | | | | | | | | | | | | | | |
| Herba Asari | x | x | x | | | x | | | | | | x | x | | | | x | x | x | x | x |
| cum Radice | | | | | | | | | | | | | | | | | | | | | |
| Radix Paeoniae | x | x | x | x | x | x | | x | x | | x | x | | | | | | x | x | x | x |
| Lactiflorae | | | | | | | | | | | | | | | | | | | | | |
| Scolopendra | | | x | | x | | x | | | x | | x | | x | x | x | x | x | x | | x |
| Subspinipes | | | | | | | | | | | | | | | | | | | | | |
| Buthus | | | x | | x | x | | | | | x | x | | x | x | | x | x | | x | x |
| Martensi | | | | | | | | | | | | | | | | | | | | | |
| Myrrha | | | | | x | | | | x | x | | x | x | x | | | x | x | x | | |
| Herba | | | | | x | | | x | | | | | x | | | | | | | x | |
| Lycopodii | | | | | | | | | | | | | | | | | | | | | |
| Gummi | | | | | | | | | x | | | | | | | | | | | | |
| Olibanum | | | | | | | | | | | | | | | | | | | | | |
| Cortex | | | | x | | | | | | x | x | x | x | | x | x | x | x | x | x | |
| Eucommiae | | | | | | | | | | | | | | | | | | | | | |
| Ulmoidis | | | | | | | | | | | | | | | | | | | | | |
| Radix Ligustici | | | | x | | x | x | x | x | x | x | | x | x | | | x | x | x | | x |
| Wallichii | | | | | | | | | | | | | | | | | | | | | |
| Radix | | | | | | | | | | x | | | | | | | x | | | | |
| Pseudoginseng | | | | | | | | | | | | | | | | | | | | | |
| Rhizoma | x | x | x | x | x | x | x | x | | | | x | | | | | | | | | |
| Gastrodiae | | | | | | | | | | | | | | | | | | | | | |
| Elatae | | | | | | | | | | | | | | | | | | | | | |
| Cornu cervi | x | x | x | | x | | | | | | x | x | | | | | | x | | x | |
| Parvum | | | | | | | | | | | | | | | | | | | | | |
| Rhizoma | | | | x | | x | x | | | | x | | | | | x | | | | x | |
| Drynariae | | | | | | | | | | | | | | | | | | | | | |
| Herba Epimedii | | | | X | x | x | | x | | | x | | | | | | x | | | | |
| Chinese Pyrola | | | | | | | | | x | | | | | | | x | | | | | x |
| Herb | | | | | | | | | | | | | | | | | | | | | |
| Radix Polygoni | | | | | | | x | | | | x | | | | | | | | | | |
| Multiflori | | | | | | | | | | | | | | | | | | | | | |
| Epimedium | | | | | | | | | | x | | | | x | x | | | | | | |
| macranthum | | | | | | | | | | | | | | | | | | | | | |
| Epimedium | | | | | | | | | | | x | | | | | | x | | x | | |
| sagittatum | | | | | | | | | | | | | | | | | | | | | |
| Fructus | | | | | | x | | | | x | | | | | x | | x | | | | x |
| Psoraleae | | | | | | | | | | | | | | | | | | | | | |
| Corylifoliae | | | | | | | | | | | | | | | | | | | | | |
| Eupolyphaga | x | x | x | | x | | x | x | | | | | x | x | | x | x | x | | | |
| seu | | | | | | | | | | | | | | | | | | | | | |
| Opisthoplatia | | | | | | | | | | | | | | | | | | | | | |

In one embodiment of the invention, the compositions provided herein comprise about 0.5-99.9% of the ingredients in the composition. In a preferred embodiment, a composition for use in the present invention comprises a combination of about 30-60 mg Rhizoma Gastrodiae Elatae, about 70-105 mg Squama Manitis, about 25-40 mg Agkistrodon seu Bungarus, about 50-90 mg Radix Aconiti Lateralis Preparata, about 50-90 mg Radix Paeoniae Lactiflorae, about 25-40 mg Herba Asari cum Radice, about 70-100 mg Cornu cervi Parvum, and about 25-45 mg Eupolyphaga seu Opisthoplatia.

In another preferred embodiment, a composition for use in the present invention comprises about 30-60 mg Rhizoma Gastrodiae Elatae, about 70-105 mg Lumbricus, about 25-40 mg dry powder of Agkistrodon seu Bungarus, about 50-90 mg roots of Radix Aconiti Lateralis Preparata, about 50-90 mg Radix Paeoniae Lactiflorae, about 25-40 mg Herba Asari cum Radice, about 70-100 mg Cornu cervi Parvum, and about 25-45 mg Eupolyphaga seu Opisthoplatia.

In one embodiment of the present invention, the compositions provided herein comprise at least about 1% of Rhizoma Gastrodiae Elatae; at least about 2% of Rhizoma Gastrodiae Elatae; at least about 5% of Rhizoma Gastrodiae Elatae; at least about 8% of Rhizoma Gastrodiae Elatae; at least about 10% of Rhizoma Gastrodiae Elatae; at least about 12% of Rhizoma Gastrodiae Elatae; at least about 15% of Rhizoma Gastrodiae Elatae; at least about 20% of Rhizoma Gastrodiae Elatae; at least about 25% of Rhizoma Gastrodiae Elatae; at least about 30% of Rhizoma Gastrodiae Elatae; at least about 35% of Rhizoma Gastrodiae Elatae; at least about 40% of Rhizoma Gastrodiae Elatae; or at least about 50% of Rhizoma Gastrodiae Elatae.

In a particular embodiment, the compositions comprises at least about 2-20% of Rhizoma Gastrodiae Elatae; at least about 5-15% of Rhizoma Gastrodiae Elatae; or at least about 6%-12% of Rhizoma Gastrodiae Elatae. It shall be understood that % as disclosed herein includes w %, w/w %, v/v % and/or w/v %.

In still some embodiments, the composition provided herein comprises at least about 5 mg of Rhizoma Gastrodiae Elatae; at least about 10 mg of Rhizoma Gastrodiae Elatae; at least about 15 mg of Rhizoma Gastrodiae Elatae; at least about 20 mg of Rhizoma Gastrodiae Elatae; at least about 25 mg of Rhizoma Gastrodiae Elatae; at least about 30 mg of Rhizoma Gastrodiae Elatae; at least about 35 mg of Rhizoma Gastrodiae Elatae; at least about 40 mg of Rhizoma Gastrodiae Elatae; at least about 45 mg of Rhizoma Gastrodiae Elatae; at least about 50 mg of Rhizoma Gastrodiae Elatae; at least about 55 mg of Rhizoma Gastrodiae Elatae; at least about 60 mg of Rhizoma Gastrodiae Elatae; at least about 70 mg of Rhizoma Gastrodiae Elatae; at least about 80 mg of Rhizoma Gastrodiae Elatae; at least about 90 mg of Rhizoma Gastrodiae Elatae; at least about 100 mg of Rhizoma Gastrodiae Elatae. In a particular embodiment, the composition comprises at least about 10-100 mg of Rhizoma Gastrodiae Elatae; at least about 20-80 mg of Rhizoma Gastrodiae Elatae; at least about 30-60 mg of Rhizoma Gastrodiae Elatae or at least about 35-50 mg of Rhizoma Gastrodiae Elatae.

In one embodiment, the composition as provided herein comprises at least about 1% of Squama Manitis; at least about 2% of Squama Manitis; at least about 5% of Squama Manitis; at least about 8% of Squama Manitis; at least about 10% of Squama Manitis; at least about 12% of Squama Manitis; at least about 14% of Squama Manitis; at least about 21% of Squama Manitis; at least about 25% of Squama Manitis; at least about 30% of Squama Manitis; at least about 35% of Squama Manitis; at least about 40% of Squama Manitis; or at least about 50% of Squama Manitis. In a particular embodiment, the composition comprises at least about 2-30% of Squama Manitis; at least about 10-25% of Squama Manitis; or at least about 14%-21% of Squama Manitis.

In some embodiments, the composition provided herein comprises at least about 5 mg of Squama Manitis; at least about 10 mg of Squama Manitis; at least about 20 mg of Squama Manitis; at least about 30 mg of Squama Manitis; at least about 40 mg of Squama Manitis; at least about 50 mg of Squama Manitis; at least about 55 mg of Squama Manitis; at least about 60 mg of Squama Manitis; at least about 65 mg of Squama Manitis; at least about 70 mg of Squama Manitis; at least about 75 mg of Squama Manitis; at least about 80 mg of Squama Manitis; at least about 85 mg of Squama Manitis; at least about 90 mg of Squama Manitis; at least about 95 mg of Squama Manitis; at least about 100 mg of Squama Manitis, at least about 105 mg of Squama Manitis; at least about 110 mg of Squama Manitis; at least about 120 mg of Squama Manitis; at least about 130 mg of Squama Manitis; at least about 140 mg of Squama Manitis; or at least about 150 mg of Squama Manitis. In a particular embodiment, the composition comprises at least about 50-150 mg of Squama Manitis; at least about 60-110 mg of Squama Manitis; at least about 70-105 mg of Squama Manitis or at least about 85-100 mg of Squama Manitis.

In one embodiment, the composition as provided herein comprises at least about 1% of Lumbricus; at least about 2% of Lumbricus; at least about 5% of Lumbricus; at least about 8% of Lumbricus; at least about 10% of Lumbricus; at least about 12% of Lumbricus; at least about 14% of Lumbricus; at least about 21% of Lumbricus; at least about 25% of Lumbricus; at least about 30% of Lumbricus; at least about 35% of Lumbricus; at least about 40% of Lumbricus; or at least about 50% of Lumbricus. In a particular embodiment, the composition comprises at least about 2-30% of Lumbricus; at least about 10-25% of Lumbricus; or at least about 14%-21% of Lumbricus.

In some embodiments, the composition provided herein comprises at least about 5 mg of Lumbricus; at least about 10 mg of Lumbricus; at least about 20 mg of Lumbricus; at least about 30 mg of Lumbricus; at least about 40 mg of Lumbricus; at least about 50 mg of Lumbricus; at least about 55 mg of Lumbricus; at least about 60 mg of Lumbricus; at least about 65 mg of Lumbricus; at least about 70 mg of Lumbricus; at least about 75 mg of Lumbricus; at least about 80 mg of Lumbricus; at least about 85 mg of Lumbricus; at least about 90 mg of Lumbricus; at least about 95 mg of Lumbricus; at least about 100 mg of Lumbricus, at least about 105 mg of Lumbricus; at least about 110 mg of Lumbricus; at least about 120 mg of Lumbricus; at least about 130 mg of Lumbricus; at least about 140 mg of Lumbricus; or at least about 150 mg of Lumbricus. In a particular embodiment, the composition comprises at least about 50-150 mg of Lumbricus; at least about 60-110 mg of Lumbricus; at least about 70-105 mg of Lumbricus or at least about 85-100 mg of Lumbricus.

In one embodiment, the composition as provided herein comprises at least about 1% of Agkistrodon seu Bungarus; at least about 2% of Agkistrodon seu Bungarus; at least about 3% of Agkistrodon seu Bungarus; at least about 4% of Agkistrodon seu Bungarus; at least about 5% of Agkistrodon seu Bungarus; at least about 6% of Agkistrodon seu Bungarus; at least about 7% of Agkistrodon seu Bungarus; at least about 8% of Agkistrodon seu Bungarus; at least about 10% of Agkistrodon seu Bungarus; at least about 20% of Agkistrodon seu Bungarus; at least about 30% of Agkistrodon seu Bungarus; at least about 40% of Agkistrodon seu Bungarus; or at least about 50% of Agkistrodon seu Bungarus. In a particular embodiment, the composition comprises at least about 1-10% of Agkistrodon seu Bungarus; at least about 2-8% of Agkistrodon seu Bungarus; or at least about 5-8% of Agkistrodon seu Bungarus.

In some embodiments, the composition provided herein comprises at least about 5 mg of Agkistrodon seu Bungarus; at least about 10 mg of Agkistrodon seu Bungarus; at least about 20 mg of Agkistrodon seu Bungarus; at least about 25 mg of Agkistrodon seu Bungarus; at least about 30 mg of Agkistrodon seu Bungarus; at least about 35 mg of Agkistrodon seu Bungarus; at least about 40 mg of Agkistrodon seu Bungarus; at least about 45 mg of Agkistrodon seu Bungarus; at least about 50 mg of Agkistrodon seu Bungarus; at least about 55 mg of Agkistrodon seu Bungarus; at least about 60 mg of Agkistrodon seu Bungarus; at least about 70 mg of Agkistrodon seu Bungarus; at least about 80 mg of Agkistrodon seu Bungarus; at least about 90 mg of Agkistrodon seu Bungarus; at least about 100 mg of Agkistrodon seu Bungarus, at least about 105 mg of Agkistrodon seu Bungarus; or at least about 110 mg of Agkistrodon seu Bungarus. In a particular embodiment, the composition comprises at least about 10-100 mg of Agkistrodon seu Bungarus; at least about 15-85 mg of Agkistrodon seu Bungarus; at least about 20-50 mg of Agkistrodon seu Bungarus; at least about 25-40 mg of Agkistrodon seu Bungarus; or at least about 30-35 mg of Agkistrodon seu Bungarus.

In one embodiment, the composition as provided herein comprises at least about 1% of Radix Aconiti Lateralis Preparata; at least about 2% of Radix Aconiti Lateralis Preparata; at least about 5% of Radix Aconiti Lateralis Preparata; at least about 8% of Radix Aconiti Lateralis Preparata; at least about 10% of Radix Aconiti Lateralis Preparata; at least about 12% of Radix Aconiti Lateralis Preparata; at least about 14% of Radix Aconiti Lateralis Preparata; at least about 18% of Radix Aconiti Lateralis Preparata; at least about 25% of Radix Aconiti Lateralis Preparata; at least about 30% of Radix Aconiti Lateralis Preparata; at least about 35% of Radix Aconiti Lateralis Preparata; at least about 40% of Radix Aconiti Lateralis Preparata; or at least about 50% of Radix Aconiti Lateralis Preparata. In a particular embodiment, the composition comprises at least about 2-30% of Radix Aconiti Lateralis Preparata; at least about 5-25% of Radix Aconiti Lateralis Preparata; or at least about 10-18% of Radix Aconiti Lateralis Preparata.

In some embodiments, the composition provided herein comprises at least about 5 mg of Radix Aconiti Lateralis Preparata; at least about 10 mg of Radix Aconiti Lateralis Preparata; at least about 20 mg of Radix Aconiti Lateralis Preparata; at least about 30 mg of Radix Aconiti Lateralis Preparata; at least about 40 mg of Radix Aconiti Lateralis Preparata; at least about 50 mg of Radix Aconiti Lateralis Preparata; at least about 55 mg of Radix Aconiti Lateralis Preparata; at least about 60 mg of Radix Aconiti Lateralis Preparata; at least about 65 mg of Radix Aconiti Lateralis Preparata; at least about 70 mg of Radix Aconiti Lateralis Preparata; at least about 75 mg of Radix Aconiti Lateralis Preparata; at least about 80 mg of Radix Aconiti Lateralis Preparata; at least about 85 mg of Radix Aconiti Lateralis Preparata; at least about 90 mg of Radix Aconiti Lateralis Preparata; at least about 95 mg of Radix Aconiti Lateralis Preparata; at least about 100 mg of Radix Aconiti Lateralis Preparata, at least about 105 mg of Radix Aconiti Lateralis Preparata; at least about 110 mg of Radix Aconiti Lateralis Preparata; at least about 120 mg of Radix Aconiti Lateralis Preparata; at least about 130 mg of Radix Aconiti Lateralis Preparata; at least about 140 mg of Radix Aconiti Lateralis Preparata; or at least about 150 mg of Radix Aconiti Lateralis Preparata. In a particular embodiment, the composition comprises at least about 10-150 mg of Radix Aconiti Lateralis Preparata; at least about 20-100 mg of Radix Aconiti Lateralis Preparata; at least about 50-90 mg of Radix Aconiti Lateralis Preparataor at least about 60-80 mg of Radix Aconiti Lateralis Preparata.

In one embodiment, the composition as provided herein comprises at least about 1% of Radix Paeoniae Lactiflorae; at least about 2% of Radix Paeoniae Lactiflorae; at least about 5% of Radix Paeoniae Lactiflorae; at least about 8% of Radix Paeoniae Lactiflorae; at least about 10% of Radix Paeoniae Lactiflorae; at least about 12% of Radix Paeoniae Lactiflorae; at least about 14% of Radix Paeoniae Lactiflorae; at least about 18% of Radix Paeoniae Lactiflorae; at least about 25% of Radix Paeoniae Lactiflorae; at least about 30% of Radix Paeoniae Lactiflorae; at least about 35% of Radix Paeoniae Lactiflorae; at least about 40% of Radix Paeoniae Lactiflorae; or at least about 50% of Radix Paeoniae Lactiflorae. In a particular embodiment, the composition comprises at least about 2-30% of Radix Paeoniae Lactiflorae; at least about 5-25% of Radix Paeoniae Lactiflorae; or at least about 10-18% of Radix Paeoniae Lactiflorae.

In some embodiments, the composition provided herein comprises at least about 5 mg of Radix Paeoniae Lactiflorae; at least about 10 mg of Radix Paeoniae Lactiflorae; at least about 20 mg of Radix Paeoniae Lactiflorae; at least about 30 mg of Radix Paeoniae Lactiflorae; at least about 40 mg of Radix Paeoniae Lactiflorae; at least about 50 mg of Radix Paeoniae Lactiflorae; at least about 55 mg of Radix Paeoniae Lactiflorae; at least about 60 mg of Radix Paeoniae Lactiflorae; at least about 65 mg of Radix Paeoniae Lactiflorae; at least about 70 mg of Radix Paeoniae Lactiflorae; at least about 75 mg of Radix Paeoniae Lactiflorae; at least about 80 mg of Radix Paeoniae Lactiflorae; at least about 85 mg of Radix Paeoniae Lactiflorae; at least about 90 mg of Radix Paeoniae Lactiflorae; at least about 95 mg of Radix Paeoniae Lactiflorae; at least about 100 mg of Radix Paeoniae Lactiflorae, at least about 105 mg of Radix Paeoniae Lactiflorae; at least about 110 mg of Radix Paeoniae Lactiflorae; at least about 120 mg of Radix Paeoniae Lactiflorae; at least about 130 mg of Radix Paeoniae Lactiflorae; at least about 140 mg of Radix Paeoniae Lactiflorae; or at least about 150 mg of Radix Paeoniae Lactiflorae. In a particular embodiment, the composition comprises at least about 10-150 mg of Radix Paeoniae Lactiflorae; at least about 20-100 mg of Radix Paeoniae Lactiflorae; at least about 50-90 mg of Radix Paeoniae Lactiflorae or at least about 60-80 mg of Radix Paeoniae Lactiflorae.

In one embodiment, the composition as provided herein comprises at least about 1% of Herba Asari cum Radice; at least about 2% of Herba Asari cum Radice; at least about 3% of Herba Asari cum Radice; at least about 4% of Herba Asari cum Radice; at least about 5% of Herba Asari cum Radice; at least about 6% of Herba Asari cum Radice; at least about 7% of Herba Asari cum Radice; at least about 8% of Herba Asari cum Radice; at least about 10% of Herba Asari cum Radice; at least about 20% of Herba Asari cum Radice; at least about 30% of Herba Asari cum Radice; at least about 40% of Herba Asari cum Radice; or at least about 50% of Herba Asari cum Radice. In a particular embodiment, the composition comprises at least about 1-10% of Herba Asari cum Radice; at least about 2-9% of Herba Asari cum Radice; or at least about 5-8% of Herba Asari cum Radice.

In some embodiments, the composition provided herein comprises at least about 5 mg of Herba Asari cum Radice; at least about 10 mg of Herba Asari cum Radice; at least about 20 mg of Herba Asari cum Radice; at least about 25 mg of Herba Asari cum Radice; at least about 30 mg of Herba Asari cum Radice; at least about 35 mg of Herba Asari cum Radice; at least about 40 mg of Herba Asari cum Radice; at least about 45 mg of Herba Asari cum Radice; at least about 50 mg of Herba Asari cum Radice; at least about 55 mg of Herba Asari cum Radice; at least about 60 mg of Herba Asari cum Radice; at least about 70 mg of Herba Asari cum Radice; at least about 80 mg of Herba Asari cum Radice; at least about 90 mg of Herba Asari cum Radice; at least about 100 mg of Herba Asari cum Radice, at least about 105 mg of Herba Asari cum Radice; or at least about 110 mg of Herba Asari cum Radice. In a particular embodiment, the composition comprises at least about 10-100 mg of Herba Asari cum Radice; at least about 15-85 mg of Herba Asari cum Radice; at least about 20-50 mg of Herba Asari cum Radice; at least about 25-40 mg of Herba Asari cum Radice; or at least about 30-35 mg of Herba Asari cum Radice.

In one embodiment, the composition as provided herein comprises at least about 1% of Cornu cervi Parvum; at least about 5% of Cornu cervi Parvum; at least about 8% of Cornu cervi Parvum; at least about 10% of Cornu cervi Parvum; at least about 12% of Cornu cervi Parvum; at least about 14% of Cornu cervi Parvum; at least about 20% of Cornu cervi Parvum; at least about 25% of Cornu cervi Parvum; at least about 30% of Cornu cervi Parvum; at least about 35% of Cornu cervi Parvum; at least about 40% of Cornu cervi Parvum; or at least about 50% of Cornu cervi Parvum. In a particular embodiment, the composition comprises at least about 2-30% of Cornu cervi Parvum; at least about 10-25% of Cornu cervi Parvum; or at least about 14%-20% of Cornu cervi Parvum.

In some embodiments, the composition provided herein comprises at least about 5 mg of Cornu cervi Parvum; at least about 10 mg of Cornu cervi Parvum; at least about 20 mg of Cornu cervi Parvum; at least about 30 mg of Cornu cervi Parvum; at least about 40 mg of Cornu cervi Parvum; at least about 50 mg of Cornu cervi Parvum; at least about 55 mg of Cornu cervi Parvum; at least about 60 mg of Cornu cervi Parvum; at least about 65 mg of Cornu cervi Parvum; at least about 70 mg of Cornu cervi Parvum; at least about 75 mg of Cornu cervi Parvum; at least about 80 mg of Cornu cervi Parvum; at least about 85 mg of Cornu cervi Parvum; at least about 90 mg of Cornu cervi Parvum; at least about 95 mg of Cornu cervi Parvum; at least about 100 mg of Cornu cervi Parvum, at least about 105 mg of Cornu cervi Parvum; at least about 110 mg of Cornu cervi Parvum; at least about 120 mg of Cornu cervi Parvum; at least about 130 mg of Cornu cervi Parvum; at least about 140 mg of Cornu cervi Parvum; or at least about 150 mg of Cornu cervi Parvum. In a particular embodiment, the composition comprises at least about 50-150 mg of Cornu cervi Parvum; at least about 60-110 mg of Cornu cervi Parvum; at least about 70-100 mg of Cornu cervi Parvum or at least about 80-90 mg of Cornu cervi Parvum.

In one embodiment, the composition as provided herein comprises at least about 1% of Eupolyphaga seu Opisthoplatia; at least about 2% of Eupolyphaga seu Opisthoplatia; at least about 3% of Eupolyphaga seu Opisthoplatia; at least about 4% of Eupolyphaga seu Opisthoplatia; at least about 5% of Eupolyphaga seu Opisthoplatia; at least about 6% of Eupolyphaga seu Opisthoplatia; at least about 7% of Eupolyphaga seu Opisthoplatia; at least about 8% of Eupolyphaga seu Opisthoplatia; at least about 9% of Eupolyphaga seu Opisthoplatia; at least about 10% of Eupolyphaga seu Opisthoplatia; at least about 20% of Eupolyphaga seu Opisthoplatia; at least about 30% of Eupolyphaga seu Opisthoplatia; at least about 40% of Eupolyphaga seu Opisthoplatia; or at least about 50% of Eupolyphaga seu Opisthoplatia. In a particular embodiment, the composition comprises at least about 1-10% of Eupolyphaga seu Opisthoplatia; at least about 5-9% of Eupolyphaga seu Opisthoplatia; or at least about 6-8% of Eupolyphaga seu Opisthoplatia.

In some embodiments, the composition provided herein comprises at least about 5 mg of Eupolyphaga seu Opisthoplatia; at least about 10 mg of Eupolyphaga seu Opisthoplatia; at least about 20 mg of Eupolyphaga seu Opisthoplatia; at least about 25 mg of Eupolyphaga seu Opisthoplatia; at least about 30 mg of Eupolyphaga seu Opisthoplatia; at least about 35 mg of Eupolyphaga seu Opisthoplatia; at least about 40 mg of Eupolyphaga seu Opisthoplatia; at least about 45 mg of Eupolyphaga seu Opisthoplatia; at least about 50 mg of Eupolyphaga seu Opisthoplatia; at least about 55 mg of Eupolyphaga seu Opisthoplatia; at least about 60 mg of Eupolyphaga seu Opisthoplatia; at least about 70 mg of Eupolyphaga seu Opisthoplatia; at least about 80 mg of Eupolyphaga seu Opisthoplatia; at least about 90 mg of Eupolyphaga seu Opisthoplatia; at least about 100 mg of Eupolyphaga seu Opisthoplatia, or at least about 110 mg of Eupolyphaga seu Opisthoplatia. In a particular embodiment, the composition comprises at least about 10-100 mg of Eupolyphaga seu Opisthoplatia; at least about 15-85 mg of Eupolyphaga seu Opisthoplatia; at least about 20-50 mg of Eupolyphaga seu Opisthoplatia; at least about 25-45 mg of Eupolyphaga seu Opisthoplatia; or at least about 30-35 mg of Eupolyphaga seu Opisthoplatia.

Without limiting the scope of the present invention, an amount of composition of the present invention suitable for a daily dose can be equivalent to about 0.005 to 100 grams of an formulation of the invention per kilogram body weight of the subject. In one embodiment, an amount of formulation of the present invention suitable for a daily dose can be equivalent to about 0.02 to about 50 grams per kilogram body weight of the subject. In one particular embodiment of the invention, the formulation is in a form of a pill of about 0.5 g in weight. The tablet can be administered to the subject many times in a day. In one embodiment, the dosage is two pills each time twice a day.

c. Other Ingredients

In one embodiment of the present invention, the composition of the invention is either administered along with a pain medicine or is formulated together with a pain medicine. In case of a composition for alleviating physical discomfort where the composition is given to the subject as a dietary supplement, the composition may be given simultaneously or sequentially with a pain medicine. In case of a composition for alleviating a pain-related condition where the composition is given to a subject as a prescribed drug or over the counter drug, the composition may be given simulataneously or sequentially with a pain medicine or formulated together with a pain medicine and given as one dose such as a pill. In one embodiment, the pain medicine can be without limitation, a NSAID, analgesic or anesthetic. In a preferred embodiment, the pain medicine is NSAID. In another embodiment, the composition herein comprises about 0.5-60% of the NSAID.

Various examples of NSAID include, but are not limited to, salicylates such as, aspirin, amoxiprin, benorilate, choline magnesium salicylate, diflunisal, faislamine, methyl salicylate, and salicyl salicylate (salsalate); arylalkanoic acids such as, diclofenac, aceclofenac, acemetacin, bromfenac, etodolac, indometacin, ketorolac, nabumetone, sulindac, and tolmetin; 2-arylpropionic acids (profens) such as, ibuprofen, carprofen, ffenbufen, fenoprofen, flurbiprofen, ketoprofen, loxoprofen, naproxen, tiaprofenic acid; N-arylanthranilic acids (fenamic acids) such as, mefenamic acid, meclofenamic acid, and tolfenamic acid; pyrazolidine derivatives such as, phenylbutazone, azapropazone, metamizole, and oxyphenbutazone; oxicams such as, piroxicam, lomoxicam, meloxicam, and tenoxicam; coxibs such as, celecoxib, rofecoxib, valdecoxib, etoricoxib, lumiracoxib, and parecoxib; sulphonanilides such as, nimesulide; and others such as licofelone and omega-3 fatty acids. In a preferred embodiment, the NSAID includes sulindac.

In one embodiment, the composition for alleviating pain-related conditions as provided herein comprises at least about 1% of NSAID; at least about 2% of NSAID; at least about 3% of NSAID; at least about 4% of NSAID; at least about 5% of NSAID; at least about 6% of NSAID; at least about 7% of NSAID; at least about 8% of NSAID; at least about 9% of NSAID; at least about 10% of NSAID; at least about 20% of NSAID; or at least about 30% of NSAID. In a particular embodiment, the composition comprises at least about 1-10% of NSAID; at least about 5-9% of NSAID; or at least about 6-8% of NSAID.

In some embodiments, the composition for alleviating pain-related conditions as provided herein comprises at least about 5 mg of NSAID; at least about 10 mg of NSAID; at least about 20 mg of NSAID; at least about 25 mg of NSAID; at least about 30 mg of NSAID; at least about 35 mg of NSAID; at least about 40 mg of NSAID; at least about 45 mg of NSAID; at least about 50 mg of NSAID; at least about 55 mg of NSAID; at least about 60 mg of NSAID; at least about 70 mg of NSAID; at least about 80 mg of NSAID; at least about 90 mg of NSAID; at least about 100 mg of NSAID, or at least about 110 mg of NSAID. In a particular embodiment, the composition comprises at least about 10-100 mg of NSAID; at least about 15-85 mg of NSAID; at least about 20-50 mg of NSAID; at least about 25-45 mg of NSAID; or at least about 30-35 mg of NSAID.

In one embodiment, the composition for alleviating physical discomfort as provided herein can be administered sequentially or simultaneously with at least about 10-100 mg of NSAID; at least about 15-85 mg of NSAID; at least about 20-50 mg of NSAID; at least about 25-45 mg of NSAID; or at least about 30-35 mg of NSAID.

Without limiting the scope of the present invention the combination of the ingredients as provided herein can be used in combination with other pain medicines such as analgesics and anaesthetics. Examples of analgesics include but are not limited to, acetaminophen, aspirin, bupivacain, boprenorphine, butorphanol, celecoxib, clofenadol, choline, clonidine, codeine, diflunisal, dihydrocodeine, dibydroergotamine, dihydromorphine, ethylmorphine, etodolac, eletriptan, eptazocine, ergotamine, fentanyl, fentoprofen, hyaluronic acid, hydrocodon, hydromorphon, hylan, ibuprofen, lindomethacin, ketorolac, lcetotifen, levomethadon, levallorphan, levorphanol, lidocaine, mefenamic acid, meloxicam, meperidine, metl1 adone, morphine, nabumetone, nalbuphin, nefopam, nalorphine, naloxone, naltrexone, naproxen, naratriptan, nefazodone, mormethadon, oxaprozin, oxycodone, oxymorphon, pentazocin, pethidine, phenpyramid, piritramid, piroxicam, propoxyphene, refecoxib, rizatriptan, salsalaketoprofen, sulindac, sumatriptan, tebacon, tilidin, tolmetin, tramadol, zolmitriptan and their salts.

It is within the scope of the present invention to optionally add at least one mineral to the compositions as provided herein. The minerals include but are not limited to, one or more of calcium, phosphorus, cobalt, copper, iron, manganese, zinc and selenium. Calcium helps in the maintenance of bones and teeth, in cell communication, regulation of body, regulation of enzymes and in blood clotting. Calcium can be involved in transmitting chemical and electrical signals along nerves and muscles, and may help in the release of neurotransmitters, which allow nerve impulses to pass from one nerve to another and from nerves to tissues. Calcium can be helpful in blood pressure regulation, and signaling the secretion of substances that regulate blood pressure. Inside muscle cells, calcium can allow two muscle proteins, actin and myosin, to interact to cause muscle contraction. Phosphorus is a component of phospholipids, which form the structure of cell membranes. Phosphorus is a constituent of DNA and RNA; is involved in regulating enzyme activity; and is a buffer that helps regulate the pH in the cytoplasm of all cells. The composition may contain at least about 0.5% of the minerals in the formulation. The minerals can be obtained commercially.

It is within the scope of the present invention to optionally add at least one vitamin and/or enzyme to the formulations as provided herein. The vitamins include the vitamins known in the art including but are not limited to, vitamin A, vitamin D, vitamin E, Vitamin B12, riboflavin, niacin, pantothenic acid, thiamine, choline, folic acid, biotin, vitamin K and vitamin C. The composition may contain at least about 0.5% of the vitamins in the formulation. The vitamins can be obtained commercially. The formulations as provided herein may optionally contain at least one enzyme. Examples of enzymes include, but are not limited to, bromelain, papain, fungal proteases, acid stable proteases, neutral stable proteases, and alkaline stable proteases. Enzymes useful in the invention can be derived from any source such as porcine, bovine, fungi, or plants. Any method known in the art can be used to obtain an enzyme. For example, standard protein isolation techniques can be used to obtain an enzyme preparation. In addition, enzymes can be obtained commercially. For example, enzymes can be obtained from National Enzyme Company (Forsyth, Mont.), American Laboratories. Incorporated (Omaha, Nebr.), Botanical International (Long Beach, Calif.), or Marcor Development Corporation (Carlstadt, N.J.). The composition may contain at least about 0.5% of the enzyme in the formulation.

It is within the scope of the present invention to optionally add one or more amino acids, glucosamine, acetylglucosamine, chondroitin sulfate, radical scavengers, antioxidants, reducing agents, or mixtures thereof, to the formulation provided herein. Examples of radical scavengers and antioxidants include, without limitation, ascorbic acid, tocopheryl acetate, tocopheryl palmitate, tocopherol, and butyl hydroxytoluene. A composition can contain any amount of radical scavengers, antioxidants, reducing agents, or mixtures thereof. For example, at least 0.5% of the formulation as provided herein can be a radical scavenger, antioxidant, reducing agent, or mixture thereof. In one embodiment of the invention, the herbal formula of the invention further comprises, one or more of ingredients selected from natural or artificial flavor, color, mineral, vitamin, enzyme, amino acids, glucosamine, acetylglucosamine, chondroitin sulfate, radical scavengers, antioxidants, preservative, or combinations thereof.

Various examples of other natural ingredients that can be used in the composition of the present invention include, but are not limited to, Achyranthes and Cyathula Root (Radix Achyranthis Bidentatae et Radix Cyathulae), Argyi Leaf (Folium Artemisiae Argyi), Aucklandia Root (Radix Aucklandiae), Bomeol (Bomeolum), Chuanxiong Rhizome (Rhizoma Ligustici Chuanxiong), Cinnamon Bark (Cortex Cinnamomi), Corydalis Tuber (Rhizoma Corydalis), Curcuma Root (Radix Curcumae), Dangshen (Radix Codonopsis Pilosulae), Evodia Fruit (Fructus Evodiae), Ledebouriella Root (Radix Ledebouriellae), Light Wheat (Fructus Tritici Levis), Notoginseng (Radix Notoginseng), Pilose Antler (Cornu Cervi Pantotrichum), Safflower (Flos Carthami), and Tortoise Plastron (Plastrum Testudinis).

### B. Methods of Use

One aspect of the present invention provides a method of alleviating physical discomfort in a subject using the composition for alleviating physical discomfort, as provided herein. In another aspect the present invention provides a method of alleviating a pain-related condition in a subject using the composition for alleviating a pain-related condition, as provided herein. As described above, the ingredients in the compositions have a synergistic effect that can yield unexpected therapeutic benefits for alleviating physical discomfort or pain-related conditions in a subject. The ingredients in the compositions provided herein increase blood circulation thereby increasing the efficiency with which the ingredients can be delivered through soft tissues, to joints in order to reduce the inflammation and control pain. Increase in blood circulation itself can also effectively reduce inflammation, thus alleviating pain related conditions, such as, musculoskeletal or nerve pain. In addition, delivery of the combination of ingredients that stimulate bone health can achieve a long term curative effect. The enhancement of the blood circulation by the combination of ingredients as provided herein, can also be effectively used to treat muscle cramping or stiffness that may be caused by excessive exercise or stress by reducing inflammation. Therefore, the combination of the ingredients to produce a synergistic effect can unexpectedly enhance the beneficial effect of the individual ingredient/s and improve the overall health of the subject.

In a preferred embodiment, the invention provides a method of alleviating a pain-related condition in a subject by administering to a subject an effective amount of a formulation comprising a combination of ingredients or extracts therefrom comprising Rhizoma Gastrodiae Elatae, Squama Manitis, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia.

In another preferred embodiment, the invention provides a method of alleviating a pain-related condition in a subject by administering to a subject an effective amount of a formulation comprising a combination of ingredients or extracts therefrom comprising Rhizoma Gastrodiae Elatae, Lumbricus, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia.

In one embodiment, the formulations used in the methods of the invention further comprise one or more of NSAIDs. In one embodiment, the NSAID is selected from aspirin, paracetamol, amoxiprin, benorilate, choline magnesium salicylate, diflunisal, faislamine, methyl salicylate, salicyl salicylate (salsalate), diclofenac, aceclofenac, acemetacin, bromfenac, etodolac, indometacin, ketorolac, nabumetone, sulindac, tolmetin, ibuprofen, carprofen, ffenbufen, fenoprofen, flurbiprofen, ketoprofen, loxoprofen, naproxen, tiaprofenic acid, mefenamic acid, meclofenamic acid, tolfenamic acid, phenylbutazone, azapropazone, metamizole, oxyphenbutazone, piroxicam, lomoxicam, meloxicam, tenoxicam, celecoxib, rofecoxib, valdecoxib, etoricoxib, lumiracoxib, parecoxib, nimesulide, licofelone, omega-3 fatty acids, or combinations thereof. In a preferred embodiment, the NSAID used in the methods of the invention includes sulindac.

The combination of ingredients in the methods of the invention can be effective to treat various pain related conditions, including but are not limited to, arthritis, including OA and RA; musculoskeletal pain, or nerve-related pain. The musculoskeletal pain can include, for example, sciatica nerve pain, cervical spondylosis, tenovaginitis, ankylosing spondylitis, periarthritis of the shoulder, back and leg pain, neck, shoulder and arm pain, whiplash injuries, motor vehicle, work-related injuries, failed back surgery and other post surgical pain syndromes, cervicogenic headache, pain due to arthritis, myofascial pain, and fibromyalgia. The common symptoms of OA and RA can include steady or intermittent pain in a joint, stiffness after periods of inactivity, swelling or tenderness in 1 or more joints, etc. Low back pain can result from intervertebral disc herniation in the lower spine and pressing against a nerve causing excruciating burning pain called sciatica, and/or lumbar spinal canal stenosis.

In one preferred embodiment, the methods of the invention are used to alleviate osteoarthritis. The treatment of osteoarthritis using the composition of the present invention is exemplified in Example 2. In one embodiment, the methods of the invention are used to alleviate rheumatoid arthritis. The treatment of rheumatoid arthritis using the composition of the present invention is exemplified in Example 4. In one embodiment, the methods of the invention are used to alleviate cervical spondylosis. The treatment of cervical spondylosis using the composition of the present invention is exemplified in Example 5.

Various pain related conditions that can be treated using the combination of natural ingredients as provided herein, include, but are not limited to, musculoskeletal pain, nerve related pain, arthritis such as, rheumatoid arthritis, osteoarthritis, polyarthritis, ulnar deviation, boutonniere deformity, swan neck deformity, z-thumb deformity, subluxation at the metacarpophalangeal joint; inflammatory arthropathies, such as, ankylosing spondylitis, psoriatic arthritis, Reiter's syndrome, acute gout, dystocia, postpartum lochiostasis, abdominal pain, and metastatic bone pain. Other pain related conditions include but are not limited to, cancer related pain, vascular pain, raynaud's disease, psychogenic pain, trigeminal neuralgia, spinal cord injury, spasticity, post dural puncture headache, pelvic pain, head and neck cancer pain, complex regional pain syndrome, postherpetic neuralgia (shingles), peripheral neuralgia, nerve injuries, phantom limb pain, pelvic and urogenital pain, post-traumatic pain, post-amputation pain, temporomandibular disorders, and AIDS-related pain.

The examples of pains due to sports injury, strain or inflammation of tendons or ligaments include but are not limited to, tennis elbow, frozen shoulder, carpal tunnel syndrome, plantar fasciitis, and Achilles tendonitis. Tennis elbow can be due to inflammation of the tendons of the hand gripping muscles where these tendons are attached to the elbow. Conventional treatment includes injection of steroids at the tender spot. Frozen shoulder is a stiffening of the ligaments around the shoulder joint which may come on after prolonged unaccustomed use of the arm. Treatment includes a program of exercises to increase the range of movement of the arm with a steroid injection into the shoulder to get it moving again. Carpal tunnel syndrome involves a nerve which passes through the carpal tunnel on the front of the wrist into the human hand. When this tunnel becomes inflamed it can press on the nerve causing shooting pain into the thumb and first two fingers. The syndrome can arise due to conditions such as thyroid disease, pregnancy and arthritis. Treatments include steroid injections, surgery and rest. Plantar fasciitis involves ligaments in the sole of the foot which can get inflamed leading to pain on the bottom of the heel while walking. Steroid injections and orthotic shoe devices can be used to treat the condition. Achilles tendonitis involves the Achilles tendon located at the back of the human ankle and which becomes inflamed and painful when walking or painful to stand up on tip-toe. This condition is usually caused by shoes which rub at the back of the heel.

Without limiting the scope of the invention, the combination of ingredients as provided herein can also be used to treat the side effects of chemotherapy, treating the cravings and withdrawal symptoms of drug addicts and treating a variety of other chronic conditions, such as, diabetes, antibiotic-resistant infection etc. The combination of ingredients as provided herein can also be used by veterinarian to treat various animals. The dietary supplement of the invention can have nutritional as well as healing properties for the animals.

Yet another aspect of the invention provides a method of improving an efficacy of one or more NSAIDs in a subject, by co-administering with the one or more NSAIDs an effective amount of a formulation to the subject comprising:

(a) a combination of one or more ingredients that improve blood circulation selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba or extracts therefrom;

one or more ingredients that reduce pain selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, or extracts therefrom; and

one or more ingredients that enhance bone health selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, glucosamine, acetylglucosamine, chondroitin, calcium, phosphorus, or extracts from natural ingredients; and b) one or more of a NSAID.

In one preferred embodiment, the method of improving an efficacy of one or more NSAIDs in a subject, comprises co-administering with one or more NSAIDs an effective amount of a formulation comprising a combination of ingredients or extracts therefrom comprising Rhizoma Gastrodiae Elatae, Squama Manitis, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia; and one or more of NSAIDs.

In another preferred embodiment, the method of improving an efficacy of one or more NSAIDs in a subject, comprises co-administering with the one or more NSAIDs an effective amount of a formulation to the subject comprising a combination of ingredients or extracts therefrom comprising Rhizoma Gastrodiae Elatae, Lumbricus, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia; and one or more of NSAIDs.

The NSAID can be administered sequentially or simultaneously with the composition of the combination of the natural ingredients as provided herein. Alternatively, the NSAID can be formulated together with the combination of the natural ingredients and is given as one formulation such as, one pill.

In one embodiment of the invention, the improvement in the efficacy of one or more non-steroidal anti-inflammatory drugs comprises at least one of reduction in pain, reduction in a dosage, reduction in a side-effect, enhancement of blood circulation, or combinations thereof.

There are various adverse drug reactions (ADRs) associated with the use of NSAID. For example, patients on NSAID can also suffer from anemia, either as a consequence of the disease itself (anaemia of chronic disease) or as a consequence of gastrointestinal bleeding as a side effect of NSAID used in treatment. The ADRs associated with NSAIDs relate to gastrointestinal (GI) effects, renal effects, and cardiovascular effects of the agents. These effects can be dose-dependent, and may pose the risk of ulcer perforation, upper gastrointestinal bleeding, and death, thereby limiting the use of NSAID therapy. The ADRs associated with use of NSAIDs relate to direct and indirect irritation of the gastrointestinal tract (GIT). NSAIDs can cause a dual insult on the GIT—the acidic molecules can directly irritate the gastric mucosa; and inhibition of COX-1 can reduce the levels of protective prostaglandins. Other common gastrointestinal ADRs can include nausea, dyspepsia, gastric ulceration/bleeding, and/or diarrhea. Risk of ulceration can increase with duration of therapy, and with higher doses. In attempting to minimise GI ADRs, it may be desirable to use the lowest effective dose for the shortest period of time.

NSAIDs can also be associated with a relatively high incidence of renal ADRs. The mechanism of these renal ADRs can be due to changes in renal haemodynamics (bloodflow), mediated by prostaglandins, which can be affected by NSAIDs. Examples of some ADRs associated with altered renal function include but are not limited to, salt and fluid retention, and hypertension. These agents may cause further renal impairment in combination with other nephrotoxic agents such as, ACE inhibitor and a diuretic. NSAIDs may also cause other renal conditions, such as, interstitial nephritis, nephrotic syndrome, acute renal failure, and acute tubular necrosis.

NSAIDs can also be associated with a relatively high incidence of cardiovascular risk. The NSAID can results in myocardial infarction, heart attack, or stroke.

Photosensitivity can be another adverse effect of many of the NSAIDs. Examples of other ADRs include but are not limited to, raised liver enzymes, headache, dizziness, heart failure, hyperkalaemia, confusion, bronchospasm, rash, and irritable bowel syndrome symptoms.

In one embodiment, the reduction in the side effects of the NSAID in the methods of the invention is selected from gastrointestinal effect, renal effect, cardiovascular effect, photosensitivity, raised liver enzymes, headache, dizziness, hyperkalaemia, confusion, bronchospasm, rash, irritable bowel syndrome symptoms, or combinations thereof.

The combination of the ingredients administered along with the NSAID as provided herein, can result in the enhancement of the blood circulation, thereby increasing the therapeutic efficacy of the NSAID in alleviating pain related conditions such as, nerve or musculoskeletal pain. By combination with the ingredients of the invention, NSAID may be delivered/penetrated to tissues, where otherwise it may not be reached. This could provide additional therapeutic benefits that may not be provided by NSAID alone. The enhancement of the blood circulation and the ease of penetration of the ingredients to the joints can help increase the effectiveness of NSAID and thereby reduce the dosage of the NSAID needed. The reduced dosage of NSAID can thereby reduce potential adverse affects associated with high dosage of NSAID.

### C. Process of Manufacture

### 1. Extraction Methods

Another aspect of the present invention provides a process of preparation of a composition useful for alleviating physical discomfort in a subject, comprising a composition for alleviating physical discomfort as disclosed herein.

Yet another aspect of the invention provides a process of preparing a composition useful for alleviating a pain-related condition in a subject, comprising a composition for alleviating a pain-related condition, as disclosed herein.

In a preferred embodiment, the process of preparing a composition useful for alleviating a pain-related condition in a subject, comprises combining Rhizoma Gastrodiae Elatae, Squama Manitis, Agkistrodon seu Bungarus, Radix Aconiti Lateralis Preparata, Radix Paeoniae Lactiflorae, Herba Asari cum Radice, Cornu cervi Parvum, Eupolyphaga seu Opisthoplatia, extracts therefrom, or combinations thereof.

In another preferred embodiment, the process of preparing a composition useful for alleviating a pain-related condition in a subject, comprises combining Rhizoma Gastrodiae Elatae, Lumbricus, Agkistrodon seu Bungarus, Radix Aconiti Lateralis Preparata, Radix Paeoniae Lactiflorae, Herba Asari cum Radice, Cornu cervi Parvum, Eupolyphaga seu Opisthoplatia, extracts therefrom, or combinations thereof.

In one embodiment, at least some of the ingredients in the process of preparation of formulation herein, are subjected to one or more prior extraction steps. In one preferred embodiment, the extraction step includes aqueous extraction step. In one embodiment, the aqueous extraction step is carried out by boiling the ingredient in water. In one preferred embodiment, the boiling is carried out for about 15-60 minutes. In another embodiment, the aqueous extract is subjected to a concentration step. In one embodiment, the aqueous extract is dried or allowed to dry. In another embodiment, the ingredients themselves are dried or allowed to dry. In one embodiment, the dried ingredients are grinded to powder. In one embodiment, the size of the powder is smaller than 200 mesh. In one preferred embodiment, the size of the powder is between 150-200 mesh.

The natural ingredients can be obtained commercially in the form of a powder or in their respective natural state such as a root or a stem etc. The extraction of the natural ingredients can comprise reducing the size of the herbal materials (for example by pulverizing) followed by extraction by heating with a suitable solvent such as water or alcohol. The extracts can then be concentrated and/or dried by natural evaporation or vacuum rotary evaporation. At any point in the process, the intermediate products of any step may be subjected to concentration, clarification or purification steps. Thereafter, some of the extracts can further be purified and tested for improved efficacy.

For example, in one embodiment, Rhizoma Gastrodiae Elatae, dry powder of Manitis, dry powder of Agkistrodon seu Bungarus, roots of Radix Aconiti Lateralis Preparata and Radix Paeoniae Lactiflorae, Herba Asari cum Radice, Cornu cervi Parvum, and Eupolyphaga seu Opisthoplatia, can be pulverized to a coarse powder. The ingredients can then be refluxed together with water or ethanol for 0.5-1 hour each. The water extracts can then be combined, filtered, and concentrated to the concentration of 100% (w/v) by vacuum evaporation to produce extract. The natural ingredients can optionally be extracted by supercritical fluid extraction (SFE) technique with supercritical carbon dioxide to produce extract. Alternatively, each natural ingredient can be separately refluxed with water or ethanol for 0.5-1 hour each. Each water extract can then be filtered, and concentrated to the concentration of 100% (w/v) by vacuum evaporation to produce extract. Each natural ingredient can be optionally extracted by supercritical fluid extraction (SFE) technique with supercritical carbon dioxide to produce extract. All the extracts can then be mixed to make one extract with a combination of natural ingredients.

In another embodiment, the natural ingredients can be extracted by soaking with 0.1% hydrochloric acid solution (pH 2.0) for 12-24 hours each, at room temperature. The hydrochloric acid extracts can then be combined, filtered, and adjusted to pH 6.0 with 1% sodium hydroxide solution, then concentrated to the concentration of 100% (w/v) by vacuum evaporation to produce extract. The criterias for determining the optimal extraction process can be maximal extractions of known active ingredients and maximal beneficial pharmacological effects.

In one embodiment, the extraction process can include providing the dried herb, acidifying the dried herb by soaking it in rice vinegar, heating the acid-soaked herb to dryness, and decocting the herb in water, repeating the decoction, combining the filtrates from these two decoctions, and concentrating the filtrates to form an extract. The extract may then be further processed to render it more suitable for oral administration. As for the acidification step, various kinds of edible acids besides naturally fermented vinegar may be used. For example, artificially synthesized acetic acid or natural fruit juices may be used to acidify the herb. In addition, any suitable bone from other animals such as pigs, reptiles, mammals or fish may be used.

The extraction process can also comprise firstly, soaking a raw composition such as, Rhizoma Gastrodiae Elatae, dry powder of Manitis, dry powder of Agkistrodon seu Bungarus, roots of Radix Aconiti Lateralis Preparata and Radix Paeoniae Lactiflorae, Herba Asari cum Radice, Cornu cervi Parvum, and Eupolyphaga seu Opisthoplatia in a predetermined amount of water for a predetermined period of soaking time to form a pre-decocting solution and a pre-decocting composition. The time period for soaking can be from half an hour to 24 hours or more. Secondly, heating the pre-decocting solution and the pre-decocting composition for a predetermined period of reaction time to form a mixture consisting of a residual composition and a solution; and thirdly, cooling down the solution to a predetermined temperature. Additionally, a person skilled in the art will recognize that although the decoction can be concentrated under reduced pressure and the extract can be obtained by spray drying, it is equally feasible to obtain the extract by lyophilization or freeze drying.

The extraction process entails reducing the size of the herbal materials. The reduction in size may be achieved by a number of ways including, but are not limited to, cutting, chopping, mincing, pounding, pulverizing, milling and grinding. While one way may be taught, other ways and means of achieving a reduction in size of the materials may also be used.

According to traditional Chinese medicine practice, dried plant materials are typically preferred for use. Thus, all weights referring to natural ingredients herein can be the dry weights of those herbs. However, drying may not be a requirement to derive the benefits of these herbs. As such, the present invention may be practiced with the listed fresh plant materials as well. The use of fresh plant materials, sufficient to meet the requisite quantity and proportions of the extracts used, come under the scope of the present invention. It is understood that several species within a plant genus may be given under a particular plant's entry and these species with the same genus may be freely substituted by, or used in conjunction with, other members of the same genus as given in the present invention. The herb material may need to be processed before the extraction process. The processing methods include but are not limited to: discarding miscellaneous substances to make the herbs clean, cutting and/or breaking into small pieces, dissolving the effective components into a liquid substance, stir-frying, steaming, quenching, fermenting, etc.

In addition, it is recognized that certain plant parts may contain the active ingredients of interest in higher concentration and the present invention may teach the use of specific plant parts, however, these ingredients may also be present in the other parts of the same plants. As such, the ingredients of interest may also be extracted from other parts of the same plant under the scope of the present invention. A person skilled in the art will appreciate that it is possible, with plant cell and tissue culture techniques, to culture the cells and tissue of these herbs in vitro and to extract the active ingredients of interest from these cells and tissue.

### 2. Purification Process

An extract of the formulation obtained from any of the various possible processes described above, may be further purified. The extract can be purified by using one or more separation techniques in which the unwanted components can be reduced or removed, thus increasing the relative abundance of the ingredient in the extract. The ingredients can be analyzed for their purity after the purification step. The examples of the separation/analysis techniques include but are not limited to, thin layer chromatography (TLC), high performance liquid chromatography (HPLC), liquid chromatography-mass spectrometry (LC-MS), gas chromatography-LC (GC-LC), gas chromatography-mass spectrometry (GC-MS), nuclear magnetic resonance (NMR), infrared spectroscopy (IR), x-ray crystallography, fluorescence spectroscopy, UV-vis, and assays. Additionally, chromatographies of ion exchange, normal and reverse phase absorption can also be used for purification of active ingredients.

The natural ingredients composing the composition as provided herein, can be identified by an Ingredient Profiling Method using liquid chromatography-mass spectrometry (LC-MS-MS). Abundent active ingredients in each composition can be purified using the methods described above.

### 3. Quality Control Standards

For quality control, the assay tests can be used to detect the presence of some of the usable markers or major ingredients of the formulation can be determined and profiled by TLC analysis, LC-MS/MS method or other analytic methods. For example, an ingredient can be pulverized to a coarse powder. The ingredient can then be refluxed together with water or alcohol. The extracts can then be filtered, and subject to analysis. The most abundant components (for example, top 10 of most abundant components) in the ingredient can be determined and quantified by TLC or LC-MS/MS analysis. This can be used as a profile or “fingerprint” for this ingredient. Once profiles for all ingredients are established, these profiles will be used as references for each ingredient to ensure the quality of each ingredient.

Additionally, various separation/analysis techniques such as described above, can be used as analytical methods for identification of different ingredients in the formulation. Chromatographic fingerprint analysis technique can be used to assess the quality of the composition of the present invention by the comparison of its chromatographic fingerprint with that of the standard specimen of the ingredient.

### 4. Packaging Process

The ingredients of the present invention can be packaged as a capsule for oral administration. As such the dried ingredients can be grinded to powders, and then mixed into capsules. Alternatively, the extract of the ingredients can be precipitated and then used to fill capsule shells. Another method that can be used is to co-precipitate the extracts, either individually or with other extracts, with suitable excipients, before filling capsule shells. In one embodiment, dextrin, soluble starch and/or aspartame as excipients can be mixed with the extract in a mixer. Sufficient liquid ethanol (75%) may be added as a binder. This admixture can be mixed well and then vacuum dried in a dryer. The admixture can then be granulated by a granulator to obtain a fine powder. Talcum powder may be added to improve flowability before capsules are filled with the extract powder for oral administration. The unpackaged extract can be used for the animal studies while the extract in capsules can be given to human patients for the clinical observations. In another embodiment, the extract can be further mixed with one or more ingredients such as, a NSAID to obtain a formulation.

In one embodiment, the composition of the present invention can be packaged in a form of a kit. By way of example only, the kit may include a storage and transport tube for storing the individual ingredients of the present invention or alternatively, the tube may contain the combination of the ingredients of the present invention. The tube can be glass or plastic and the tube may have a replaceable end closure. The kit may also include written instructions for making and using the formulation. In some embodiments, NSAID can be provided as separate composition in separate containers within the kit for the treatment. Suitable packaging and additional articles for use (e.g., measuring cup for liquid preparations, foil wrapping to minimize exposure to air, and the like) are known in the art and may be included in the kit.

### D. Pharmaceutical Formulations and Routes of Administration

In yet another aspect of the present invention, the invention provides a pharmaceutical composition comprising composition of the invention as herein along with a pharmaceutically acceptable excipient.

In a particular embodiment, the invention provides a pharmaceutical composition for alleviating a pain-related condition in a subject, comprising a combination of:

(a) one or more ingredients that improve blood circulation selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba, xanthinol nicotinate, 3,5-pyridinedicarboxylic acid, amlodipine, prazosin, doxazosin, nitroglycerin, sildenafil, L-arginine, or extracts from natural ingredients;

one or more ingredients that reduce pain selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, morphine, diamorphine, fentanyl, buprenorphine, oxycodone, codeine, aspirin, ibuprofen, diclofenac, celecoxib, or extracts from natural ingredients; and

one or more ingredients that enhance bone health selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, bisphosphonates, glucosamine, acetylglucosamine, chondroitin, calcium, phosphorus, or extracts from natural ingredients; and

(b) one or more of NSAIDs.

In a preferred embodiment, a pharmaceutical composition for alleviating a pain-related condition in a subject, comprises a combination of ingredients or extracts therefrom comprising Rhizoma Gastrodiae Elatae, Squama Manitis, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia, and one or more of NSAIDs.

In another preferred embodiment, a pharmaceutical composition for alleviating a pain-related condition in a subject, comprises a combination of ingredients or extracts therefrom comprising Rhizoma Gastrodiae Elatae, Lumbricus, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia; and one or more of NSAIDs.

The compositions of the present invention can be delivered directly or in pharmaceutical compositions along with suitable carriers or excipients, as is well known in the art. Present methods of treatment can comprise administration of an effective amount of the composition as provided herein to a subject in need; e.g., a human having arthritis. In a preferred embodiment, the subject is a mammalian subject, and in a most preferred embodiment, the subject is a human.

An effective amount of the composition as provided herein can readily be determined by routine experimentation, as can the most effective and convenient route of administration, and the most appropriate formulation. Various formulations and drug delivery systems are available in the art. See, e.g., Gennaro, A. R., ed. (1995) Remington's Pharmaceutical Sciences, supra.

Suitable routes of administration may include, for example, oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated and mode of delivery, along with the physical, chemical, and biological properties of the ingredient, dictate the type of composition and the route of administration to be used.

In one embodiment, the administration in the methods of the invention is selected from oral administration, transmucosal administration, buccal administration, nasal administration, parental administration, intravenous, subcutaneous, intramuscular, sublingual, transdermal administration, or rectal administration. In a preferred embodiment, the administration is oral.

The formulations of the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms can be composed of the drug, an excipient(s), and a container/closure system.

The composition can be further included into drinking water to formulate a sports drink to alleviate pain-related conditions such as, inflammation caused by excessive exercise. Alternatively, the composition can be added to fruit juices, or food stuff such as, sports bars, cereal, gum, health improving food and the like. The composition of the present invention is also suitable to be used in a feed for animals such as, cats, dogs, horses, pigs, and livestock to prevent inflammatory and rheumatic arthritic diseases.

One or multiple pharmaceutically acceptable excipients can be added to the composition of the invention to improve or facilitate manufacturing, stability, administration, and safety of the ingredient/s, and can provide a means to achieve a desired ingredient release profile. Therefore, the type of excipient(s) to be added to the composition can depend on various factors, such as, for example, the physical and chemical properties of the ingredients, the route of administration, and the manufacturing procedure. Pharmaceutically acceptable excipients are available in the art and include those listed in various pharmacopoeias. (See, e.g., the U.S. Pharmacopeia (USP), Japanese Pharmacopoeia (JP), European Pharmacopoeia (EP), and British pharmacopeia (BP); the U.S. Food and Drug Administration (www.fda.gov) Center for Drug Evaluation and Research (CEDR) publications, e.g., Inactive Ingredient Guide (1996); Ash and Ash, Eds. (2002) Handbook of Pharmaceutical Additives, Synapse Information Resources, Inc., Endicott N.Y.; etc.)

Pharmaceutical or nutritional supplement dosage forms of the formulations of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the formulations of the present invention can include one or more physiologically acceptable inactive ingredients that facilitate processing of active ingredients into preparations for pharmaceutical or nutritional use.

Formulation can be dependent upon the desired route of administration. In one embodiment, the compositions of the invention are formulated into a tablet, pill, syrup, edible food, sports drink, solutions, suspensions, emulsions, ointments, gels and patches, liposomes, dragees, soft or hard shell capsules, amorphous or crystalline powders, aerosols, or lyophilized formulations. In a preferred embodiment, the formulation is in the form of a tablet, pill or capsule.

For intravenous injection, for example, the formulation may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, optionally containing penetration enhancers. Such penetrants are generally known in the art. For oral administration, the compounds can be formulated in liquid or solid dosage forms, and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

The formulations of the present invention may be manufactured in tablet, liquid, or powder form. In powder form, the various dry, powdered ingredients can be mixed together until a relatively homogeneous mixture is obtained. The powder can be generally administered by mixing with a liquid, such as water or fruit juice, and then drinking the resulting suspension. In liquid form, the powder can be mixed with an appropriate liquid carrier, preferably water, and the resulting suspension can be packaged in appropriate containers. In tablet form, the ingredients can be mixed together and then the tablets can be prepared according to methods well known in the art: (1) the wet-granulation method, (2) the dry-granulation method, or (3) direct compression. See Remington's Pharmaceutical Sciences.

Solid oral dosage forms can be obtained using excipients, which may include fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, can be used to coat tablets, resulting in dragees. When a capsule is preferred over a tablet, the ingredient powder, suspension, or solution thereof can be delivered in a compatible hard or soft shell capsule. Tablets can be discoid in shape, round, oval, oblong, cylindrical, or triangular. They may differ in size and weight depending on the amounts of ingredients present and the intended method of administration.

Binders can be added to the composition to impart cohesive qualities to the powdered material. Binders can impart a cohesiveness to the tablet formulation, which insures the tablet remaining intact after compression, as well as improving the free-flowing qualities by the composition of granules of desired hardness and size. Materials commonly used as binders include starch, gelatin, sugars such as sucrose, glucose, dextrose, molasses, and lactose, natural and synthetic gums such as gum acacia, sodium alginate, extract of Irish moss, panwar gum, ghatti gum, mucilage of isapol husks, carboxymethylcellulose, methylcellulose, polyvinylpyrrolidone, Veegum, microcrystalline cellulose, microcrystalline dextrose, amylose, and larch arabogalactan, and the like.

Lubricants can be added to the composition to improve the rate of flow of the tablet granulation, preventing adhesion of the tablet material to the surface of the dies and punches, reducing interparticle friction, and facilitating the ejection of the tablets from the die cavity. Commonly used lubricants include talc, magnesium stearate, calcium stearate, stearic acid, and hydrogenated vegetable oils. Lubricants can range from about 0.1% by weight to about 5% by weight.

Disintegrators can be added to the composition to facilitate the breakup or disintegration of tablets after administration. Materials serving as disintegrants are such as, starches, clays, celluloses, algins, or gums. Other disintegrators include Veegum HV, methylcellulose, agar, bentonite, cellulose and wood products, natural sponge, cation-exchange resins, alginic acid, guar gum, citrus pulp, cross-linked polyvinylpyrrolidone, carboxymethylcellulose, and the like.

In one embodiment, the compositions of the invention further comprise a natural or artificial flavor and/or color.

Coloring agents can be added to the composition to give tablets a more pleasing appearance, help the manufacturer to control the product during its preparation and help the user to identify the product. Any of the approved certified water-soluble FD&C dyes, mixtures thereof, or their corresponding lakes may be used to color tablets. A color lake can be a combination by adsorption of a water-soluble dye to a hydrous oxide of a heavy metal, resulting in an insoluble form of the dye.

Flavoring agents can be added to the composition to give the tablet a more palatable taste. Flavoring agents can be esters, alcohols, and aldehydes to carbohydrates and complex volatile oils. Synthetic flavors of a desired type are now available and are well known in the art.

In one embodiment, the formulation of the present invention can be administered topically, such as through a skin patch, a semi-solid, or a liquid formulation, for example a gel, a (micro-) emulsion, an ointment, a solution, a (nano/micro)-suspension, or a foam. The penetration of the ingredient into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of the ingredient of the invention. Transdermal or topical administration may be preferred, for example, in situations in which local delivery with minimal systemic exposure is desired.

For administration by inhalation, or administration to the nose, the formulation of the present invention can be delivered in the form of a solution, suspension, emulsion, or semisolid aerosol from pressurized packs, or a nebuliser, typically with the use of a propellant, e.g., halogenated carbons derived from methane and ethane, carbon dioxide, or any other suitable gas. For topical aerosols, hydrocarbons like butane, isobutene, and pentane can be useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the ingredient/s and a suitable powder base such as lactose or starch.

Formulation of the present invention for parenteral administration by injection can be sterile and, can be presented in unit dosage forms, e.g., in ampoules, syringes, injection pens, or in multi-dose containers, the latter usually containing a preservative. The composition may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral composition would be reconstituted or diluted prior to administration. Depot formulations, providing controlled or sustained release of a ingredient/s of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection of the formulation of the invention are well-known in the art and include water-based solutions containing a base, such as, for example, sodium hydroxide, to form an ionized compound, sucrose or sodium chloride as a tonicity agent, and the buffer containing phosphate or histidine. Co-solvents, such as, for example, polyethylene glycols, may be added. These water-based systems can be effective at dissolving ingredients of the invention and produce low toxicity upon systemic administration. The proportions of the components of a solution system may be varied, without destroying solubility and toxicity characteristics. Furthermore, the identity of the components may be varied. For example, the components include low-toxicity surfactants, such as polysorbates or poloxamers, polyethylene glycol or other co-solvents, biocompatible polymers such as polyvinyl pyrrolidone, and other sugars and polyols may substitute for dextrose.

A therapeutically effective dose or a nutrition supplement dose can be estimated using a variety of techniques well-known in the art. Initial doses used in animal studies may be based on effective concentrations established in cell culture assays. Dosage ranges appropriate for human subjects can be determined, for example, using data obtained from animal studies and cell culture assays.

An effective amount, a beneficial amount or a therapeutically effective amount of a formulation of the invention can refer to that amount of the ingredient that results in alleviation of symptoms. Such amount of the formulation or pharmaceutical composition as provided herein can elicit the biological or medical response of a tissue, system, animal or human that is being sought by the researcher, veterinarian, medical doctor or other clinician.

Toxicity and therapeutic efficacy of such ingredients can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. Ingredients that exhibit high therapeutic indices are preferred.

Dosages can fall within a range of circulating concentrations that includes the ED50 with little or no toxicity. Dosages may vary within this range depending upon the dosage form employed and/or the route of administration utilized. The exact formulation, route of administration, dosage, and dosage interval can be chosen according to methods known in the art, in view of the specifics of a subject's condition.

Dosage amount and interval may be adjusted individually to provide plasma levels of the active moiety that are sufficient to achieve the desired effects; i.e., the minimal effective concentration (MEC). The MEC can vary for each ingredients as provide herein but can be estimated from, for example, in vitro data and animal experiments. Dosages necessary to achieve the MEC can depend on individual characteristics and route of administration. In cases of local administration or selective uptake, the effective local concentration of the ingredient may not be related to plasma concentration. The amount of ingredient or formulation administered may be dependent on a variety of factors, including the sex, age, and weight of the subject being treated, the severity of the affliction, the manner of administration, and the judgment of the prescribing physician.

The present formulations may, if desired, be presented in as a kit, a pack or dispenser device containing one or more unit dosage forms containing the active ingredient. Such a kit, pack or device may, for example, comprise metal or plastic foil, such as a blister pack, or glass, and rubber stoppers such as in vials. The kit, pack or dispenser device may be accompanied by instructions for administration. Compositions comprising ingredient/s of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein and are specifically contemplated.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### Example 1

### Treatment of Chronic Sciatica Nerve Pain by a Combination of Natural Ingredients

A female (70 years old) has had a history of sciatica nerve pain diagnosed five years ago. The acute nerve pain became severe chronic low back pain that was uncomfortable and affected daily activity (walking difficulty). Particularly, she had difficulty in standing without support every morning at least one month before the treatment. Two pills (0.5 g each) of the composition of the present invention were taken by the female every morning and at night (twice a day). Each pill was composed of Rhizoma Gastrodiae Elatae (˜30-60 mg), dry powder of Manitis (˜70-105 mg), dry powder of Agkistrodon seu Bungarus (˜25-40 mg), roots of Radix Aconiti Lateralis Preparata (˜50-90 mg) and Radix Paeoniae Lactiflorae (˜50-90 mg), Herba Asari cum Radice (˜25-40 mg), Cornu cervi Parvum (˜70-100 mg), and Eupolyphaga seu Opisthoplatia (˜25-45 mg).

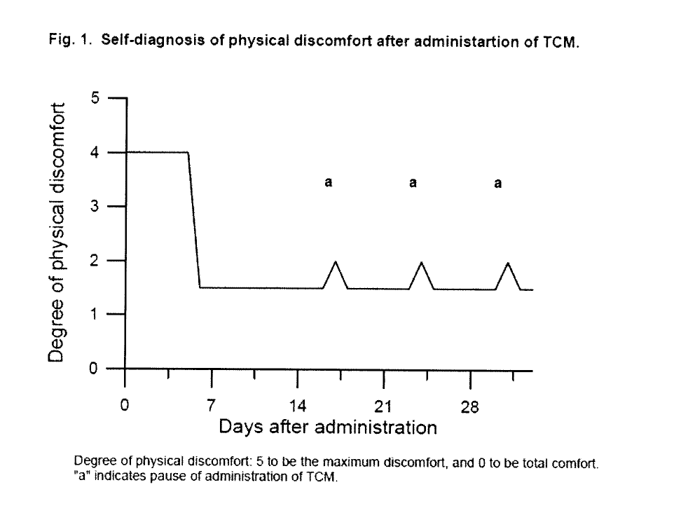

Self diagnosis indicated that discomfort decreased at approximately fifth day after taking the pills (FIG. 1) indicating the effectiveness of the pills. The period for the drug to take effect is considered as short as for any TCM, in which it often requires as long as two weeks or more for building the effectiveness. The noticeable improvements in the female patient included: reduction of low back pain to a minimum level, ease in standing up each morning without any support, and increase of daily activity (increase of walking distance from ˜2500 steps to ˜7000 steps daily). After three-month treatment, the subject was able to sit on her knees (a traditional Japanese style of sitting). The subject voluntarily took the pills for more than one and half year, and the condition remains stable. The results indicate that the composition is effective for the treatment of sciatica nerve pain.

### Example 2

### Treatment of Osteoarthritis by a Combination of Natural Ingredients

A female subject (61 years old), who was diagnosed as a mild osteoarthritis patient, had experienced stiffness on fingers every morning for more than one year. The joint of both hands showed characteristic arthritis deterioration determined by X-ray examination. After taking two pills composed of Rhizoma Gastrodiae Elatae (˜30-60 mg), dry powder of Manitis (˜70-105 mg), dry powder of Agkistrodon seu Bungarus (˜25-40 mg), roots of Radix Aconiti Lateralis Preparata (˜50-90 mg) and Radix Paeoniae Lactiflorae (˜50-90 mg), Herba Asari cum Radice (˜25-40 mg), Cornu cervi Parvum (˜70-100 mg), and Eupolyphaga seu Opisthoplatia (˜25-45 mg), every morning and night for one week, the stiffness reportedly disappeared and condition maintained for another week under the treatment. The stiffness returned 2-days after stopping the treatment, indicating that the control of stiffness is correlated to the effect of the pills, thus, providing evidence that the composition is effective to treat osteoarthritis. The subject has been voluntarily continuing the treatment for more than a year after the experiment period.

### Example 3

### Treatment of Low Back Pain by a Combination of Natural Ingredients and Sulindac

A female subject (44 years old) had acute sciatica nerve pain 18 months ago. MRI confirmed a herniated disc at the low fifth disc. Pain extended to left leg and she was not able to walk for a week. The acute pain was managed by a combination of pain killer medication and a series of physical therapies at that time. However, chronic low back pain remained ever since. Celebrex was prescribed to the female patient but was not effective in reducing the pain. The patient was also subjected to a series of disc decompression treatments (20 treatment sessions). However, no noticeable improvement was found.

One month before taking the composition of the present invention, the patient was diagnosed for lumbar spinal canal stenosis by X-ray. In addition to chronic low back pain, the patient was uncomfortable in walking and sitting for an extended period of time. No medication and other treatment were applied prior to taking the composition of the present invention. Two pills (0.5 g each) of the composition of the present invention were taken by the patient every morning and at night (twice a day). Each pill was composed of Rhizoma Gastrodiae Elatae (˜30-60 mg), dry powder of Manitis (˜70-105 mg), dry powder of Agkistrodon seu Bungarus (˜25-40 mg), roots of Radix Aconiti Lateralis Preparata (˜50-90 mg) and Radix Paeoniae Lactiflorae (˜50-90 mg), Herba Asari cum Radice (˜25-40 mg), Cornu cervi Parvum (˜70-100 mg), Eupolyphaga seu Opisthoplatia (˜25-45 mg), and Sulindac (30 mg). Total daily dosage of Sulindac was 120 mg, which is lower than recommended dosage (400 mg/day). Sulindac is a NSAID for the treatment of arthritis, but not proposed for the treatment of sciatica nerve pain.

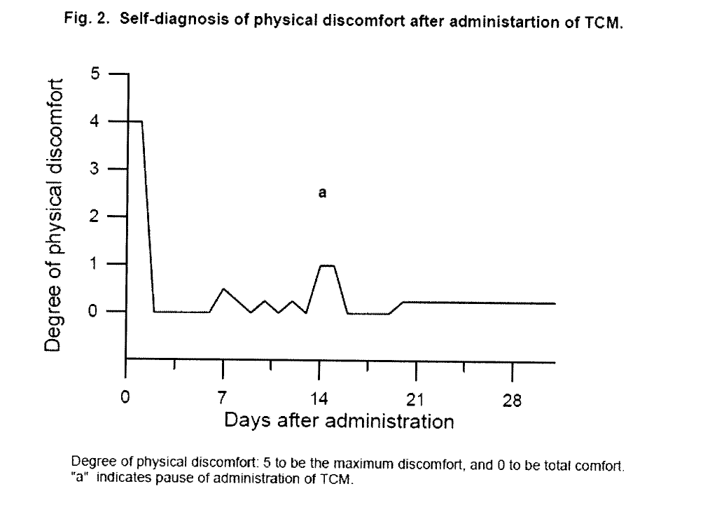

Pain reduced in the patient dramatically after first administration and condition maintained for two weeks (FIG. 2). Daily activity measured by walking distance returned to normal, i.e., before suffering from the acute sciatica crisis. Pain reemerged gradually after temporary pausing of the administration, indicating that the pain was controlled by the medication solely. After one-month of treatment, the subject was given the composition of the present invention without Sulindac, and the condition of the subject showed that the composition without sulindac was sufficient to maintain the pain control. The subject voluntarily took the pills for more than a year, and the condition remained stable. After one year treatment, the subject was able to play golf on a regular base, without taking the pills. The results clearly indicate that a composition of the present invention with NSAIDs can achieve immediate reduction of musculoskeletal and nerve pain, and improve the efficacy of NSAIDs in controlling pain by reducing its dosage.

### Example 4

### A Combination of Herbal Composition and Sulindac for Controlling Rheumatoid Arthritis

A female subject (67 years old) was diagnosed for rheumatoid arthritis for five years, and experienced pain on hands and knees. In particular, she had difficulty in standing and walking without support prior to administration of the composition of the present invention. Two pills (0.5 g each) were administered every morning and night. Each pill was composed by Rhizoma Gastrodiae Elatae (approximately 30-60 mg), dry powder of Manitis (˜70-105 mg), dry powder of Agkistrodon seu Bungarus (˜25-40 mg), roots of Radix Aconiti Lateralis Preparata (˜50-90 mg) and Radix Paeoniae Lactiflorae (˜50-90 mg), Herba Asari cum Radice (˜25-40 mg), Cornu cervi Parvum (˜70-100 mg), Eupolyphaga seu Opisthoplatia (˜25-45 mg), and Sulindac (30 mg). Total daily dosage of Sulindac was 120 mg, which is lower than recommended dosage for treating rheumatoid arthritis (400 mg/day). Pain reduced dramatically in the female subject after one week of administration. The subject was able to stand and walk freely without support after three-month administration of the composition and the condition maintained ever since. The subject has been voluntarily taking the pills for more than two years. The example indicates that the composition is effective for controlling rheumatoid arthritis.

### Example 5

### A Herbal Composition Effective for Reducing Neck Pain

A female subject (48 years old) suffered from severe neck pain for more than 3 years where the pain was caused by cervical spondylosis. The pain affected the movement of neck, shoulder, upper back, and both arms. The subject had difficulty even in using a hair drier to dry her hair. Prior to the administration of the composition of the present invention, the subject had been under going physical therapy for one year without any significant improvement. After taking two pills composed of Rhizoma Gastrodiae Elatae (approximately 30-60 mg), lumbricus (˜70-105 mg), dry powder of Agkistrodon seu Bungarus (˜25-40 mg), roots of Radix Aconiti Lateralis Preparata (˜50-90 mg), Radix Paeoniae Lactiflorae (˜50-90 mg), Herba Asari cum Radice (˜25-40 mg), Cornu cervi Parvum (70-100 mg), and Eupolyphaga seu Opisthoplatia (˜25-45 mg) every morning and night, the pain reduced dramatically. Muscle of neck and both arms were mostly relaxed. More noticeably, the subject was able to use a hair drier freely. The subject returned to normal daily life and was able to conduct most of the daily activities including playing tennis. The subject has voluntarily taken the pills continuously for more than six months.

What is claimed is:

1. A composition useful for alleviating physical discomfort in a subject, comprising one or more ingredients that improve blood circulation, one or more ingredients that reduce pain and one or more ingredients that enhance bone health.

2. The composition of claim 1 in which said physical discomfort is selected from headache, migraine, postoperative pain, ear pain, throat pain, eye pain, cutaneous pain, mild-to-moderate pain due to inflammation of tendons or ligaments, tissue injury, pain due to sports injury or strain, pyrexia, sciatic nerve pain, renal colic, hardness and stiffness of shoulder, cold constitutions, edema, frequent tiredness, insomnia, sleep difficulty due to pains, depression, dysmenorrhea, menstruation pain, amenorrhea, menopausal syndrome, muscle weakness, reduced blood circulation, weak bones, or combinations thereof.

3. The composition of claim 1 in which said one or more ingredients are selected from natural products, natural ingredients, extracts from natural ingredients, or combinations thereof.

4. The composition of claim 1 in which said one or more ingredients that improve blood circulation are selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba, or extracts therefrom.

5. The composition of claim 1 in which said one or more ingredients that reduce pain are selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, or extracts therefrom.

6. The composition of claim 1 in which said one or more ingredients that enhance bone health are selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, glucosamine, acetylglucosamine, chondroitin, calcium, phosphorus, or extracts from natural ingredients.

7. The composition of claim 1 which is administered to the subject simultaneously or sequentially with a pain medicine.

8. The composition of claim 7, wherein said pain medicine comprises a non-steroidal anti-inflammatory drug.

9. The composition of claim 8, wherein said non-steroidal anti-inflammatory drug includes sulindac.

10. A composition useful for alleviating a pain-related condition in a subject, comprising one or more ingredients that improve blood circulation, one or more ingredients that reduce pain and one or more ingredients that enhance bone health.

11. The composition of claim 10 in which said one or more ingredients are selected from synthetic compounds, natural products, natural ingredients, extracts from natural ingredients, or combinations thereof.

12. The composition of claim 10 wherein
said one or more ingredients that improve blood circulation are selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba, xanthinol nicotinate, 3,5-pyridinedicarboxylic acid, amlodipine, prazosin, doxazosin, nitroglycerin, sildenafil, L-arginine, or extracts from natural ingredients;
said one or more ingredients that reduce pain are selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, morphine, diamorphine, fentanyl, buprenorphine, oxycodone, codeine, aspirin, ibuprofen, diclofenac, celecoxib, or extracts from natural ingredients; and
said one or more ingredients that enhance bone health are selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, bisphosphonates, glucosamine, acetylglucosamine, chondroitin, calcium, phosphorus, or extracts from natural ingredients.

13. The composition of claim 10, wherein said pain-related condition is selected from musculoskeletal pain, nerve related pain, arthritis, rheumatoid arthritis, osteoarthritis, polyarthritis, ulnar deviation, boutonniere deformity, swan neck deformity, z-thumb deformity, subluxation at the metacarpophalangeal joint, inflammatory arthropathies, cervical spondylosis, ankylosing spondylitis, psoriatic arthritis, Reiter's syndrome, gout, dysmenorrhea, metastatic bone pain, cancer pain, herniated disc, tempromandibular joint dysfunction, tendonitis, torticollis, trigeminal neuralgia, cervical syndrome, thoracic outlet syndrome, complex regional pain syndrome, or combinations thereof.

14. A composition useful for alleviating a pain-related condition in a subject, comprising one or more ingredients or extract therefrom selected from Rhizoma Gastrodiae Elatae, Squama Manitis, Agkistrodon seu Bungarus, Radix Aconiti Lateralis Preparata, Radix Paeoniae Lactiflorae, Herba Asari cum Radice, Cornu cervi Parvum, or Eupolyphaga seu Opisthoplatia.

15. The composition of claim 14 which comprises about 0.5-99.9% of said ingredients or extracts therefrom.

16. A composition useful for alleviating a pain-related condition in a subject, comprising one or more ingredients or extract therefrom selected from Rhizoma Gastrodiae Elatae, Lumbricus, Agkistrodon seu Bungarus, Radix Aconiti Lateralis Preparata, Radix Paeoniae Lactiflorae, Herba Asari cum Radice, Cornu cervi Parvum, or Eupolyphaga seu Opisthoplatia.

17. The composition of claim 16 which comprises about 0.5-60% of a non-steroidal anti-inflammatory drug.

18. A process of preparing a composition useful for alleviating physical discomfort or a pain-related condition in a subject, comprising: combining
one or more ingredients that improve blood circulation selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba or extracts therefrom;
one or more ingredients that reduce pain selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, or extracts therefrom; and
one or more ingredients that enhance bone health selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, glucosamine, acetylglucosamine, chondroitin, calcium, phosphorus, or extracts from natural ingredients.

19. The process of claim 18 which further comprises combining a non-steroidal anti-inflammatory drug.

20. A method of improving an efficacy of one or more non-steroidal anti-inflammatory drugs (NSAIDs) in a subject, comprising co-administering with said one or more NSAIDs an effective amount of a formulation to a subject, the formulation comprising
(a) a combination of one or more ingredients that improve blood circulation selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba or extracts therefrom;
one or more ingredients that reduce pain selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, or extracts therefrom; and
one or more ingredients that enhance bone health selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, glucosamine, acetylglucosamine, chondroitin, calcium, phosphorus, or extracts from natural ingredients; and
b) one or more of a NSAID.

21. The method of claim 20, wherein the improvement in the efficacy of one or more non-steroidal anti-inflammatory drugs comprises at least one of reduction in pain, reduction in a dosage, reduction in a side-effect, enhancement of blood circulation, or combinations thereof.

22. The method of claim 21, wherein said side-effect is selected from gastrointestinal effect, renal effect, cardiovascular effect, photosensitivity, raised liver enzymes, headache, dizziness, hyperkalaemia, confusion, bronchospasm, rash, irritable bowel syndrome symptoms, or combinations thereof.

23. A method of improving an efficacy of one or more non-steroidal anti-inflammatory drugs (NSAIDs) in a subject, comprising: co-administering with said one or more NSAIDs an effective amount of a formulation to a subject, the formulation comprising
(a) a combination of ingredients or extracts therefrom comprising Rhizoma Gastrodiae Elatae, Squama Manitis, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia; and
(b) one or more NSAIDs.

24. A method of improving an efficacy of one or more non-steroidal anti-inflammatory drugs (NSAIDs) in a subject, comprising: co-administering with said one or more NSAIDs an effective amount of a formulation to a subject, the formulation comprising
(a) a combination of ingredients or extracts therefrom comprising Rhizoma Gastrodiae Elatae, Lumbricus, Agkistrodon seu Bungarus, Radix Aconiti Lateralis Preparata, Radix Paeoniae Lactiflorae, Herba Asari cum Radice, Cornu cervi Parvum, and Eupolyphaga seu Opisthoplatia; and
(b) one or more NSAIDs.

25. A method of alleviating physical discomfort or pain-related condition in a subject comprising: administering to a subject an effective amount of a formulation comprising a combination of
one or more ingredients that improve blood circulation selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba or extracts therefrom;
one or more ingredients that reduce pain selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, or extracts therefrom; and
one or more ingredients that enhance bone health selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, glucosamine, acetylglucosamine, chondroitin, calcium, phosphorus, or extracts from natural ingredients.

26. The method of claim 25, wherein the formulation is in a form of tablet, pill or capsule.

27. The method of claim 25, wherein the formulation is administered as a dietary supplement.

28. The method of claim 25, wherein the administration is oral administration.

29. The method of claim 25 in which said physical discomfort is selected from headache, migraine, postoperative pain, ear pain, throat pain, eye pain, cutaneous pain, mild-to-moderate pain due to inflammation of tendons or ligaments, tissue injury, pain due to sports injury or strain, pyrexia, sciatic nerve pain, renal colic, hardness and stiffness of shoulder, cold constitutions, edema, frequent tiredness, insomnia, sleep difficulty due to pains, depression, dysmenorrhea, menstruation pain, amenorrhea, menopausal syndrome, muscle weakness, reduced blood circulation, weak bones, or combinations thereof.

30. The method of claim 25, wherein said pain-related condition is selected from musculoskeletal pain, nerve related pain, arthritis, rheumatoid arthritis, osteoarthritis, polyarthritis, ulnar deviation, boutonniere deformity, swan neck deformity, z-thumb deformity, subluxation at the metacarpophalangeal joint, inflammatory arthropathies, cervical spondylosis, ankylosing spondylitis, psoriatic arthritis, Reiter's syndrome, gout, dysmenorrhea, metastatic bone pain, cancer pain, herniated disc, tempromandibular joint dysfunction, tendonitis, torticollis, trigeminal neuralgia, cervical syndrome, thoracic outlet syndrome, complex regional pain syndrome, or combinations thereof.

31. The method of claim 25, wherein the pain-related condition includes sciatic nerve pain.

32. A herbal formula that promotes cardiovascular function and a healthy circulatory system, supports cartilage and joint function, strengthens bone health, and alleviates muscle fatigue comprising:
one or more ingredients or extracts therefrom selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, or Ginko biloba;
one or more ingredients or extracts therefrom selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, or Rhizoma Gastrodiae Elatae; and
one or more ingredients or extracts therefrom selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, glucosamine, acetylglucosamine, or chondroitin.

33. The herbal formula of claim 32 which further comprises one or more of ingredients selected from natural or artificial flavor, color, mineral, vitamin, enzyme, amino acids, radical scavengers, antioxidants, preservative, or combinations thereof.

34. The herbal formula of claim 33 wherein said mineral is selected from calcium, phosphorus, cobalt, copper, iron, manganese, zinc, selenium, or combination thereof.

35. The herbal formula of claim 32 which is formulated into a tablet, pill, syrup, edible food, sports drink, solutions, suspensions, emulsions, ointments, gels and patches, liposomes, dragees, soft or hard shell capsules, amorphous or crystalline powders, aerosols, or lyophilized formulations.

36. A pharmaceutical composition for alleviating a medical condition in a subject, comprising: a combination of
(a) one or more ingredients that improve blood circulation selected from Squama Manitis, Agkistrodon seu Bungarus, Zaocys dhumnades, Lumbricus, Radix Angelicae sinensis, Radix et Caulis Jixuetent, Radix Rehmanniae Glutinosae Conquitae, Radix Puerariae, Caulis Polygoni Multiflori, Radix Clemetidis Chinesis, Radix Tripterygium wilfordii, Hydergine, Vincamine, Nimodipine, Vinpocetine, Ginko biloba, xanthinol nicotinate, 3,5-pyridinedicarboxylic acid, amlodipine, prazosin, doxazosin, nitroglycerin, sildenafil, L-arginine, or extracts from natural ingredients;
one or more ingredients that reduce pain selected from Radix Aconiti Lateralis Preparata, Herba Asari cum Radice, Radix Paeoniae Lactiflorae, Scolopendra Subspinipes, Buthus Martensi, Myrrha, Herba Lycopodii, Gummi Olibanum, Cortex Eucommiae Ulmoidis, Radix Ligustici Wallichii, Radix Pseudoginseng, Rhizoma Gastrodiae Elatae, morphine, diamorphine, fentanyl, buprenorphine, oxycodone, codeine, aspirin, ibuprofen, diclofenac, celecoxib, or extracts from natural ingredients; and
one or more ingredients that enhance bone health selected from Eupolyphaga seu Opisthoplatia, Cornu cervi Parvum, Rhizoma Drynariae, Herba Epimedii, Chinese Pyrola herb, Radix Polygoni Multiflori, Epimedium macranthum, Epimedium sagittatum, Fructus Psoraleae Corylifoliae, bisphosphonates, glucosamine, acetylglucosamine, chondroitin, calcium, phosphorus, or extracts from natural ingredients; and
(b) one or more NSAIDs.

37. The pharmaceutical composition of claim 36 which is useful for alleviating a medical condition selected from the group consisting of osteoarthritis, rheumatoid arthritis, and cervical spondylosis.

38. An herbal formula comprising one or more herbal ingredients that improve blood circulation, one or more herbal ingredients that reduce pain, one or more herbal ingredients that enhance bone health, and optionally one or more non-herbal natural or synthetic ingredients.

39. The herbal formula of claim 38 which promotes one or more of cardiovascular function and a healthy circulatory system, supports cartilage and joint function, strengthens bone health, and alleviates muscle fatigue in a subject.
